(19) United States
(12) Patent Application Publication
(10) Pub. No.: US 2011/0213260 A1
(43) Pub. Date: Sep. 1, 2011

(54) CRT LEAD PLACEMENT BASED ON OPTIMAL BRANCH SELECTION AND OPTIMAL SITE SELECTION

(75) Inventors: Allen Keel, San Francisco, CA (US); Kyungmoo Ryu, Palmdale, CA (US); Stuart Rosenberg, Castaic, CA (US); Wenbo Hou, Lancaster, CA (US); Thao Thu Nguyen, Bloomington, MN (US); Steve Koh, South Pasadena, CA (US); Kjell Norén, Solna (SE); Michael Yang, Thousand Oaks, CA (US)

(73) Assignee: PACESETTER, INC., Sylmar, CA (US)

(21) Appl. No.: 12/713,417

(22) Filed: Feb. 26, 2010

(51) Int. Cl. A61B 5/0402

(52) U.S. Cl. 600/513

(57) ABSTRACT

An exemplary method includes accessing cardiac information acquired via a catheter located at various positions in a coronary sinus of a patient where the cardiac information includes electrical information and mechanical information; calculating scores based on the cardiac information where each of the scores corresponds to the coronary sinus or a tributary of the coronary sinus; and based on the scores, selecting a tributary of the coronary sinus as an optimal candidate for placement of a left ventricular lead. Accordingly, the selected tributary may be relied on during an implant procedure for the left ventricular lead. Various other methods, devices, systems, etc., are also disclosed.

### TECHNICAL FIELD

Subject matter presented herein relates generally to electrode and lead-based investigation or therapy systems (e.g., cardiac pacing therapies, cardiac stimulation therapies, etc.).

### BACKGROUND

Cardiac resynchronization therapy (CRT) aims to improve cardiac performance by synchronizing the ventricles. While the term “synchronization” is used, for some patients, a delay between contraction of the right ventricle and the left ventricle may be optimal. Hence, the term synchronization refers more generally to ventricular timing that improves cardiac performance. A general objective measure of lack of synchrony or dyssynchrony is QRS width representative of contraction of both ventricles. For example, a QRS width greater than about 130 ms may indicate dyssynchrony.

CRT can improve a variety of cardiac performance measures including left ventricular mechanical function, cardiac index, decreased pulmonary artery pressures, decrease in myocardial oxygen consumption, decrease in dynamic mitral regurgitation, increase in global ejection fraction, decrease in NYHA class, increased quality of life scores, increased distance covered during a 6-minute walk test, etc. Effects such as reverse modeling may also be seen, for example, three to six months after initiating CRT. Patients that show such improvements are classified as CRT “responders”. However, for a variety of reasons, not all patients respond to CRT. For example, if a left ventricular stimulation lead cannot locate an electrode in a favorable position, then a patient may not respond to CRT.

Often, the ability to respond and the extent of response to CRT depends on an initial set-up of a CRT device in a patient. As described herein, various exemplary technologies aim to improve a clinician's ability to set-up a CRT at implant and to optionally optimize thereafter. In particular, various exemplary techniques are based, at least in part, on information acquired from a localization system.

### SUMMARY

An exemplary method includes accessing cardiac information acquired via a catheter located at various positions in a coronary sinus of a patient where the cardiac information includes electrical information and mechanical information; calculating scores based on the cardiac information where each of the scores corresponds to the coronary sinus or a tributary of the coronary sinus; and based on the scores, selecting a tributary of the coronary sinus as an optimal candidate for placement of a left ventricular lead. Accordingly, the selected tributary may be relied on during an implant procedure for the left ventricular lead. Various other methods, devices, systems, etc., are also disclosed.

### BRIEF DESCRIPTION OF THE DRAWINGS

Features and advantages of the described implementations can be more readily understood by reference to the following description taken in conjunction with the accompanying drawings.

FIG. 1 is a simplified diagram illustrating an exemplary implantable stimulation device in electrical communication with at least three leads implanted into a patient's heart and at least one other lead for sensing and/or delivering stimulation and/or shock therapy. Approximate locations of the right and left phrenic nerves are also shown. Other devices with more or fewer leads may also be suitable for implementation of various exemplary techniques described herein.

FIG. 2 is a functional block diagram of an exemplary implantable stimulation device illustrating basic elements that are configured to provide cardioversion, defibrillation, pacing stimulation and/or other tissue stimulation. The implantable stimulation device is further configured to sense information and administer therapy responsive to such information.

FIG. 3 is a block diagram of an exemplary scheme associated with implantation of an implantable cardiac therapy device where the scheme spans a pre-implant phase, an implant phase and a post-implant phase.

FIG. 4 is a block diagram of an exemplary method that include pre-implant planning based at least in part on acquiring position information using a localization system.

FIG. 5 is a diagram of an exemplary arrangement of leads and electrodes for acquiring data and exemplary data and metrics based on the acquired data.

FIG. 6 is a series of perspective views of an isochronal map associated with anatomy of the coronary sinus of a patient as acquired using a localization system.

FIG. 7 is an isopotential map associated with anatomy of the coronary sinus of a patient as acquired using a localization system.

FIG. 8 is a displacement or path length map associated with anatomy of the coronary sinus of a patient as acquired using a localization system.

FIG. 9 is a peak velocity map associated with anatomy of the coronary sinus of a patient as acquired using a localization system.

FIG. 10 is a block diagram of an exemplary method for ranking tributaries of the coronary sinus as candidates for placement of a lead.

FIG. 11 is a block diagram of an exemplary method for determining an optimal vein for placement of a left ventricular lead.

FIG. 12 is a block diagram of an exemplary method for determining an optimal venous site for placement of an electrode or lead.

FIG. 13 is an exemplary system for acquiring information and analyzing such information.

### DETAILED DESCRIPTION

The following description includes the best mode presently contemplated for practicing the described implementations. This description is not to be taken in a limiting sense, but rather is made merely for the purpose of describing the general principles of the implementations. The scope of the described implementations should be ascertained with reference to the issued claims. In the description that follows, like numerals or reference designators are typically used to reference like parts or elements throughout.

### Overview

Various exemplary techniques described herein pertain to analysis of electrode positions in the body. For example, during an intraoperative procedure, a clinician may maneuver an electrode-bearing catheter to various locations in one or more chambers or vessels of the heart and acquire position information sufficient to calculate one or more metrics. Various exemplary methods include performing an intraoperative procedure to acquire information to aid performance of a subsequent intraoperative procedure for placing one or more electrodes such as lead-based electrodes. For example, in a preliminary intraoperative procedure, a clinician may map the coronary sinus and regions where tributary veins join the coronary sinus. During such a procedure, the clinician may acquire electrical information, mechanical information or electrical information and mechanical information and map such information or measures derived from the information. Based on such a map, a clinician may select a tributary vein as a candidate for placement of a lead configured to deliver electrical energy to the heart (e.g., a left ventricular lead suited for CRT).

Where a selection of a tributary vein or a ranking of tributary veins has been made prior to an intraoperative procedure (e.g., per a pre-implant planning process), the actual implantation of a lead in a tributary vein may be performed more expeditiously, more optimally for delivery of a therapy, etc. Accordingly, an exemplary method can include a coronary branch selection process and an intra-branch site selection process that rely on information acquired during two or more intraoperative procedures. As described herein, an exemplary method may optionally include a branch selection process and an intra-branch site selection process during a single intraoperative procedure (e.g., during an implant procedure).

While various exemplary methods are described as being associated with a pre-implant phase, an implant phase or a post-implant phase, such methods may be optionally combined to span multiple phases and form a comprehensive method for CRT optimization.

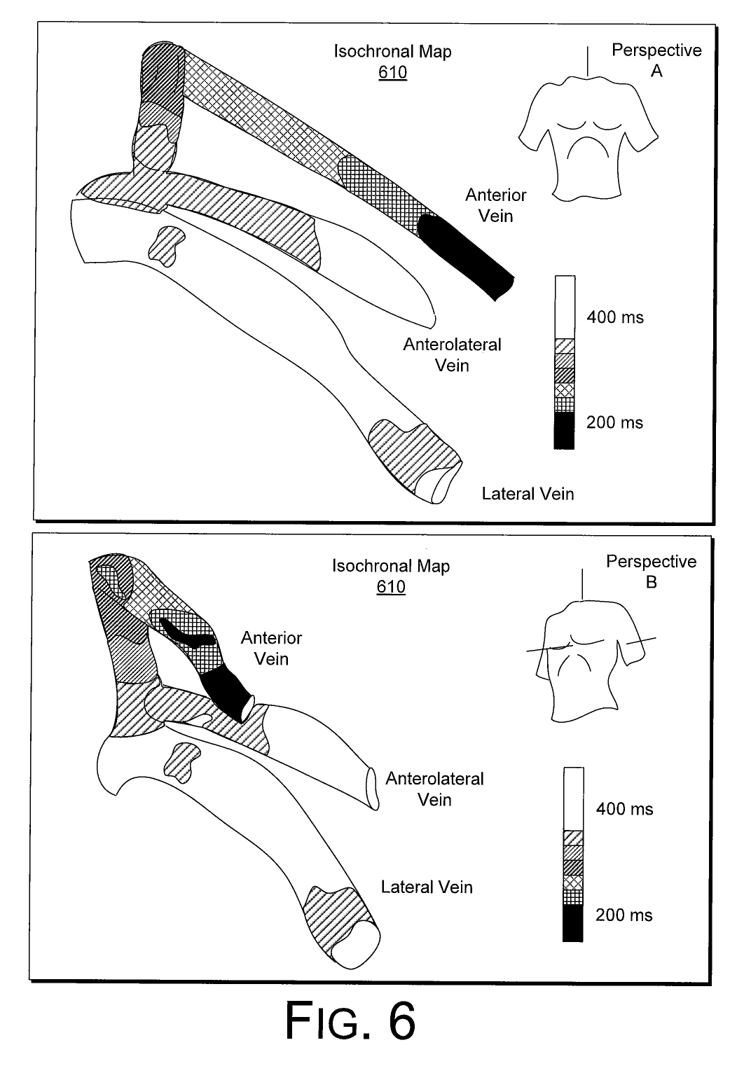

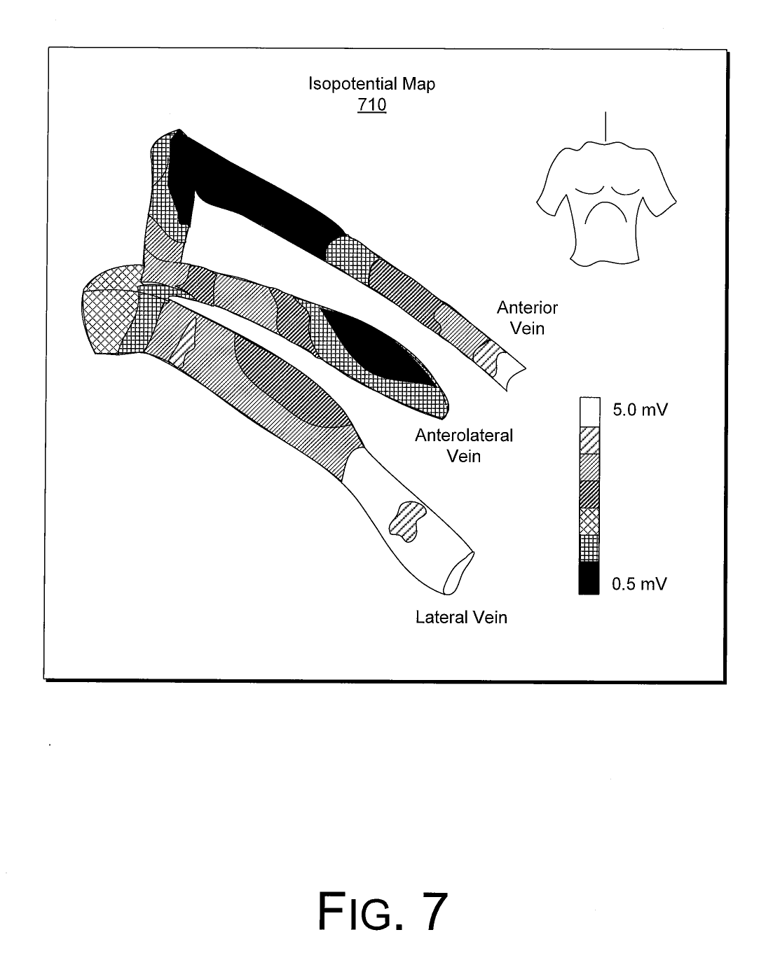

As described herein, an exemplary method may include an electrical-information-based coronary branch selection process followed by a mechanical-information-based intra-branch site selection process. In the coronary branch selection process, a clinician may insert a transvenous lead into the coronary sinus ostium and advance it to the end of the coronary sinus. An electroanatomical mapping system (e.g., a localization system such as the ENSITE® NAVX® system, St. Jude Medical Inc.) may be used to mark out the coronary sinus and cardiac veins anatomically and electrically. Resulting time-voltage maps of the coronary sinus may then allow for identification of site(s) of latest activation (see, e.g., FIG. 6) or highest voltage differential during intrinsic rhythm or right ventricular pacing (see, e.g., FIG. 7), which may be deemed as associated with the optimal coronary vein or branch for lead placement. In such a process, dispersion of electrical activation or voltage within a branch may also be used to determine an optimal coronary branch.

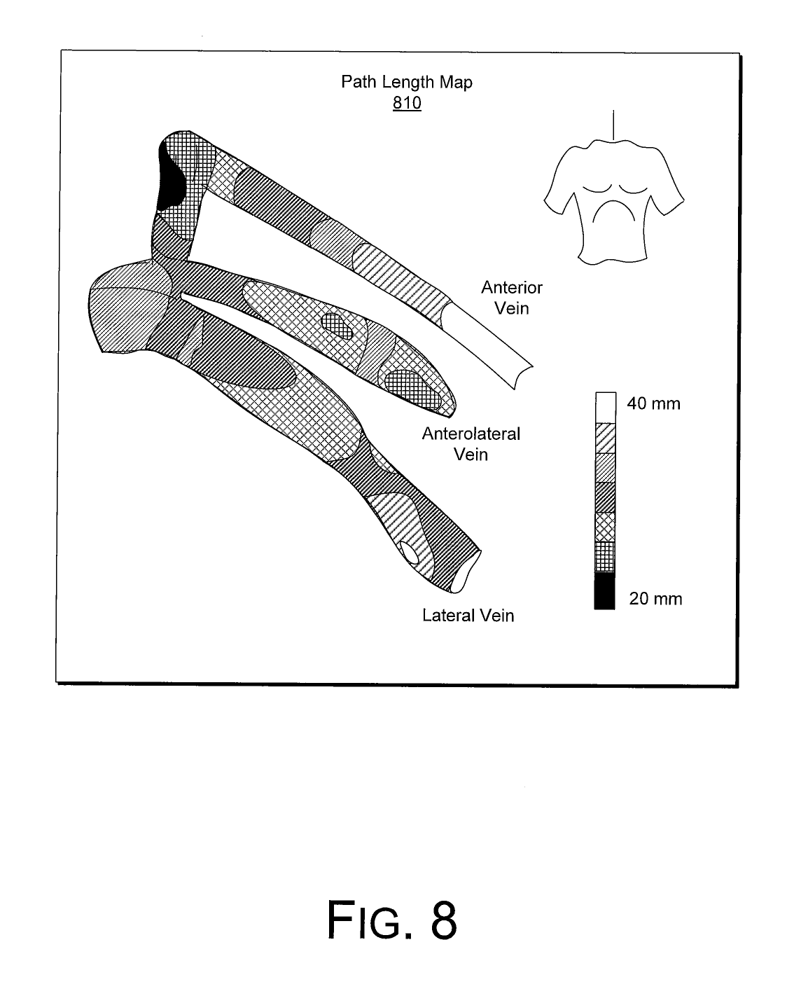

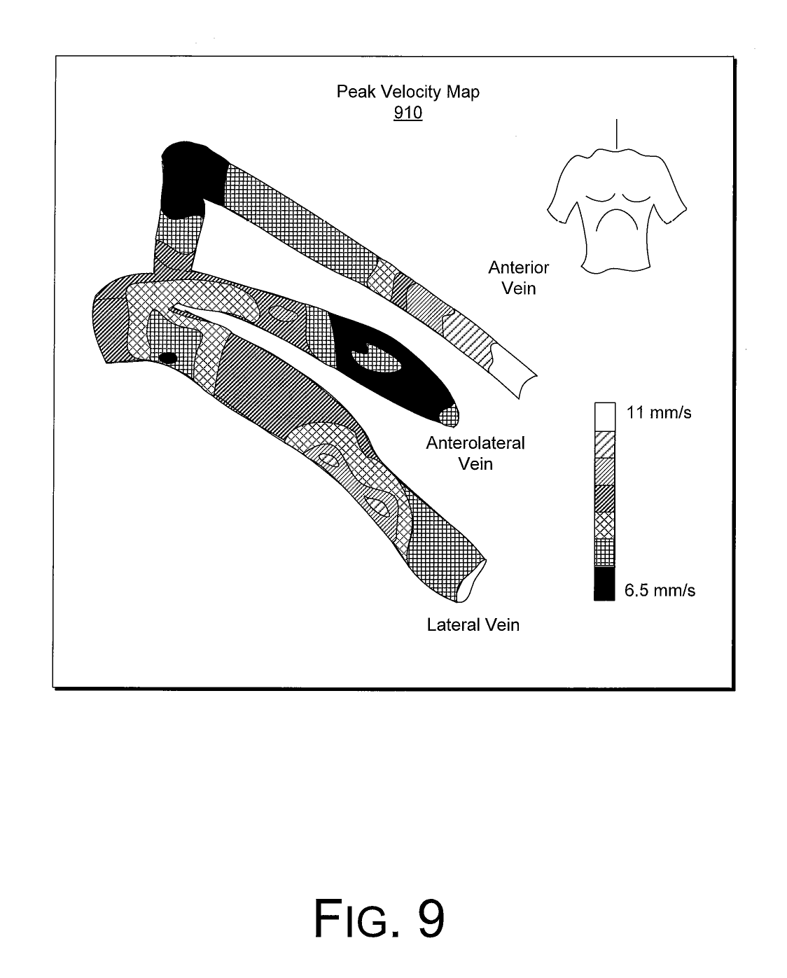

An exemplary intra-branch site selection mechanical-information-based process may, once a target vein has been selected, determine an optimal site (e.g., apically/basally) within the vein. Such a process may occur using a localization system that acquires mechanical information (e.g., to compute motion metrics for RA, RV, and LV lead electrodes). As described herein, optimal site may be determined based wholly or in part on one or more of maximum volume estimators (or metrics), minimum electromechanical delays, minimum dyssynchrony values, etc., (see, e.g., various pending U.S. patent applications, including Ser. Nos. 12/621,373; 12/398,460; 12/476,043; 12/416,771; 12/639,788; and 12/553,413 as cited in the description below, which are incorporated by reference herein. Other metrics may be used to determine an optimal site, for example, path length of each electrode (see, e.g., FIG. 8) and peak velocity of each electrode (see, e.g., FIG. 9).

With respect to pre-CRT implantation, an exemplary coronary sinus mapping technique may be applied using an EP catheter (low-Fr) during a localization study (prior to a CRT implant procedure). In addition to coronary sinus mapping, individual coronary veins/branches can be mapped. Such an extension of a coronary sinus mapping protocol can be used to provide a full activation map of the coronary venous system. Accordingly, a site of latest electrical activation circumferentially (e.g., per a coronary sinus map) may facilitate branch selection, and the site of the latest electrical activation longitudinally (e.g., via coronary vein maps) may facilitate optimal site selection within a chosen branch (basal, apical, mid-ventricular, etc).

With respect to post-CRT implantation, an exemplary coronary sinus mapping technique may be used during a standard CRT follow-up examination (e.g., 3 month or 6 month). For example, values for time from right ventricular pace to electrical activation of the left ventricular lead may examined to determine if they are more homogenous after implementation of CRT therapy. In such an example, homogeneity of values in a coronary sinus map may be used as an indicator for CRT efficacy or response.

As described herein, an exemplary system can be configured to assess motion of one or more leads in a patient's body by collected information from an implanted device (e.g., via telemetry) using, for example, a specialized localization system or an external computing device (e.g., a device programmer). Such a collection process may optionally occur at a standard CRT follow-up visit. An exemplary method includes comparing information collected post-implant to, for example, baseline information acquired pre-implant or at the time of implant. As described herein, such pre-implant information or time of implant information may be archived in memory of an implantable device or elsewhere (e.g., a database accessible by a device programmer, a localization system, etc.). Such a method may further include determining optimal settings for the implanted device (e.g., delays, electrode configuration, rates, etc.).

As described herein, an intraoperative CRT optimization process (e.g., using an localizing system) can include selecting a target coronary branch (e.g., using the site of latest electrical activation from a map of the coronary sinus) and then selecting an optimal longitudinal site within the target branch for LV lead placement (e.g., based on mechanical parameters derived from motion data collected on the RA, RV, LV lead electrodes). As described herein, such a process may be performed during two or more separate intraoperative procedures. For example, in one procedure, the coronary sinus and various coronary sinus branches can be electrically mapped with an EP catheter prior to CRT implantation to determine an optimal vein for placement of an electrode-bearing lead during a subsequent intraoperative procedure that may determine an optimal intra-branch site (e.g., optionally using a purely electrical activation-based approach). As described herein, information acquired during an intraoperative procedure may be used to assess patient health or device condition post-implant. For example, a homogeneity map of electrical activation of a patient's coronary sinus and optionally at least some of its tributaries may be used during follow-up to assess CRT efficacy.

Various exemplary methods may be implemented, for example, using a pacing system analyzer (PSA) and a localization system or a specialized localization system. Various examples are described with respect to the ENSITE® NAVX® localization system (St Jude Medical Atrial Fibrillation Division, Minnesota); noting that other types of localization systems may be used.

Various techniques aim to facilitate lead implants, particularly for leads that enter the coronary sinus to reach distal branches thereof. For example, a clinician can view plots or maps of one or more metrics and readily decide to locate a lead in a region with acceptable or optimal metrics for delivery of a cardiac therapy. A typical intraoperative, acute state process occurs iteratively (i.e., select or move, acquire, calculate; select or move, acquire, calculate; . . . ). In this iterative process, a clinician may note whether a location has acceptable metrics or unacceptable metrics.

As described herein, various exemplary techniques can be used to make decisions as to cardiac pacing therapy and optimization of a cardiac pacing therapy (e.g., CRT or other pacing therapies). In a clinical trial, acute resynchronization was shown to be a significant factor in assessing CRT efficacy and long-term outcome¹. Various methods described herein, build on this clinical finding by formulating specialized techniques and metrics associated with locations for pacing, sensing or pacing and sensing. In turn, a clinician can assess how a particular CRT therapy or configuration thereof may be expected to perform at time of implant or, in some instances, after implant. ¹G B Bleeker, S A Mollema, E R Holman, N Van De Veire, C Ypenburg, E Boersma, E E van der Wall, M J Schalij, J J Bax. “Left Ventricular Resynchronization is Mandatory for Response to Cardiac Resynchronization Therapy: Analysis in Patients with Echocardiographic Evidence of Left Ventricular Dyssynchrony at Baseline”. Circulation 2007; 116: 1440-1448.

An exemplary stimulation device is described followed by various techniques for acquiring and calculating metrics. The drawings and detailed description elucidate details of various techniques that may be used singly or in combination during an assessment or an optimization process (e.g., acute or chronic).

### Exemplary Stimulation Device

Various techniques described below are intended to be implemented in connection with any stimulation device, for example, that may be configured or configurable to delivery cardiac therapy and/or sense information germane to cardiac therapy.

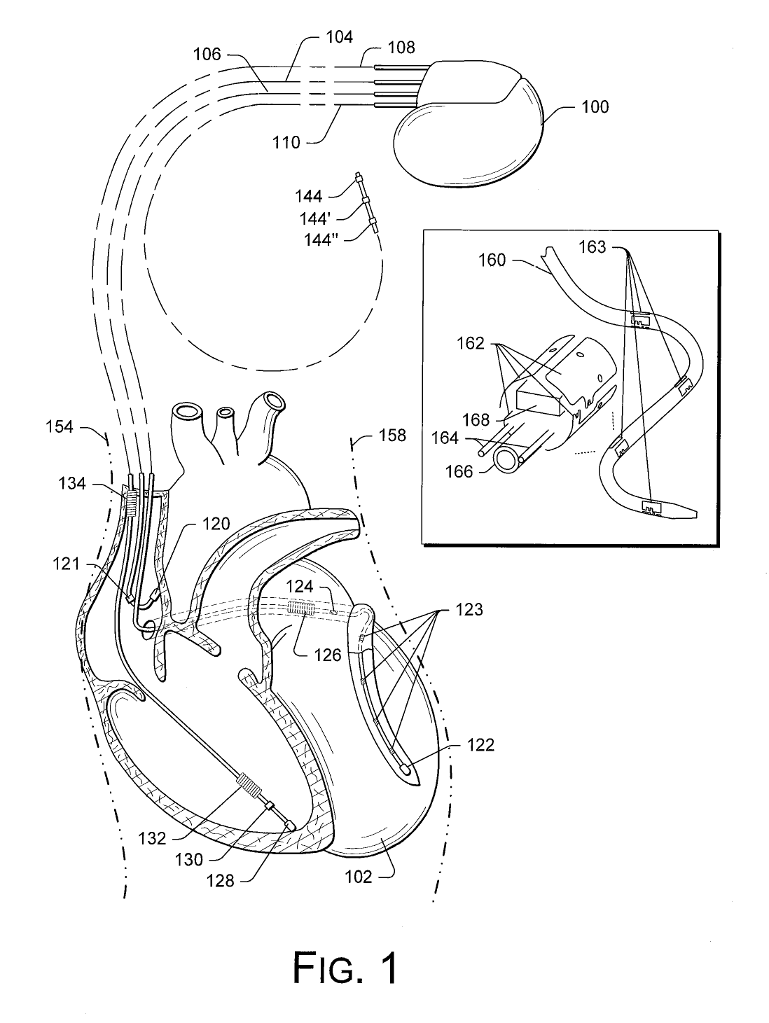

FIG. 1 shows an exemplary stimulation device 100 in electrical communication with a patient's heart 102 by way of three leads (a right atrial lead 104, a left ventricular lead 106 and a right ventricular lead 108), suitable for delivering multi-chamber stimulation and shock therapy. The leads 104, 106, 108 are optionally configurable for delivery of stimulation pulses suitable for stimulation of nerves or other tissue. In addition, in the example of FIG. 1, the device 100 includes a fourth lead 110 having multiple electrodes 144, 144′, 144″ suitable for stimulation of tissue and/or sensing of physiologic signals. This lead may be positioned in and/or near a patient's heart and/or remote from the heart.

FIG. 1 also shows approximate locations of the right and left phrenic nerves 154, 158. The phrenic nerve is made up mostly of motor nerve fibres for producing contractions of the diaphragm. In addition, it provides sensory innervation for various components of the mediastinum and pleura, as well as the upper abdomen (e.g., liver and gall bladder). The right phrenic nerve 154 passes over the brachiocephalic artery, posterior to the subclavian vein, and then crosses the root of the right lung anteriorly and then leaves the thorax by passing through the vena cava hiatus opening in the diaphragm at the level of T8. More specifically, with respect to the heart, the right phrenic nerve 154 passes over the right atrium while the left phrenic nerve 158 passes over the pericardium of the left ventricle and pierces the diaphragm separately. While certain therapies may call for phrenic nerve stimulation (e.g., for treatment of sleep apnea), in general, cardiac pacing therapies avoid phrenic nerve stimulation through judicious lead and electrode placement, selection of electrode configurations, adjustment of pacing parameters, etc.

Referring again to the various leads of the device 100, the right atrial lead 104, as the name implies, is positioned in and/or passes through a patient's right atrium. The right atrial lead 104 is configured to sense atrial cardiac signals and/or to provide right atrial chamber stimulation therapy. As described further below, the right atrial lead 104 may be used by the device 100 to acquire far-field ventricular signal data. As shown in FIG. 1, the right atrial lead 104 includes an atrial tip electrode 120, which typically is implanted in the patient's right atrial appendage, and an atrial ring electrode 121. The right atrial lead 104 may have electrodes other than the tip 120 and ring 121 electrodes. Further, the right atrial lead 104 may include electrodes suitable for stimulation and/or sensing located on a branch.

To sense atrial cardiac signals, ventricular cardiac signals and/or to provide chamber pacing therapy, particularly on the left side of a patient's heart, the stimulation device 100 is coupled to the left ventricular lead 106, which in FIG. 1 is also referred to as a coronary sinus lead as it is designed for placement in the coronary sinus and/or tributary veins of the coronary sinus. As shown in FIG. 1, the coronary sinus lead 106 is configured to position at least one distal electrode adjacent to the left ventricle and/or additional electrode(s) adjacent to the left atrium. In a normal heart, tributary veins of the coronary sinus include, but may not be limited to, the great cardiac vein, the left marginal vein, the left posterior ventricular vein, the middle cardiac vein, and the small cardiac vein.

In the example of FIG. 1, the coronary sinus lead 106 includes a series of electrodes 123. In particular, a series of four electrodes are shown positioned in an anterior vein of the heart 102. Other coronary sinus leads may include a different number of electrodes than the lead 106. As described herein, an exemplary method selects one or more electrodes (e.g., from electrodes 123 of the lead 106) and determines characteristics associated with conduction and/or timing in the heart to aid in ventricular pacing therapy and/or assessment of cardiac condition. As described in more detail below, an illustrative method acquires information using various electrode configurations where an electrode configuration typically includes at least one electrode of a coronary sinus lead or other type of left ventricular lead. Such information may be used to determine a suitable electrode configuration for the lead 106 (e.g., selection of one or more electrodes 123 of the lead 106).

In the example of FIG. 1, as connected to the device 100, the coronary sinus lead 106 is configured for acquisition of ventricular cardiac signals (and optionally atrial signals) and to deliver left ventricular pacing therapy using, for example, at least one of the electrodes 123 and/or the tip electrode 122. The lead 106 optionally allows for left atrial pacing therapy, for example, using at least the left atrial ring electrode 124. The lead 106 optionally allows for shocking therapy, for example, using at least the left atrial coil electrode 126. For a complete description of a particular coronary sinus lead, the reader is directed to U.S. Pat. No. 5,466,254, “Coronary Sinus Lead with Atrial Sensing Capability” (Helland), which is incorporated herein by reference.

The stimulation device 100 is also shown in electrical communication with the patient's heart 102 by way of an implantable right ventricular lead 108 having, in this exemplary implementation, a right ventricular tip electrode 128, a right ventricular ring electrode 130, a right ventricular (RV) coil electrode 132, and an SVC coil electrode 134. Typically, the right ventricular lead 108 is transvenously inserted into the heart 102 to place the right ventricular tip electrode 128 in the right ventricular apex so that the RV coil electrode 132 will be positioned in the right ventricle and the SVC coil electrode 134 will be positioned in the superior vena cava. Accordingly, the right ventricular lead 108, as connected to the device 100, is capable of sensing or receiving cardiac signals, and delivering stimulation in the form of pacing and shock therapy to the right ventricle. An exemplary right ventricular lead may also include at least one electrode capable of stimulating other tissue; such an electrode may be positioned on the lead or a bifurcation or leg of the lead. A right ventricular lead may include a series of electrodes, such as the series 123 of the left ventricular lead 106.

FIG. 1 also shows a lead 160 as including several electrode arrays 163. In the example of FIG. 1, each electrode array 163 of the lead 160 includes a series of electrodes 162 with an associated circuit 168. Conductors 164 provide an electrical supply and return for the circuit 168. The circuit 168 includes control logic sufficient to electrically connect the conductors 164 to one or more of the electrodes of the series 162. In the example of FIG. 1, the lead 160 includes a lumen 166 suitable for receipt of a guidewire to facilitate placement of the lead 160. As described herein, any of the leads 104, 106, 108 or 110 may include one or more electrode array, optionally configured as the electrode array 163 of the lead 160.

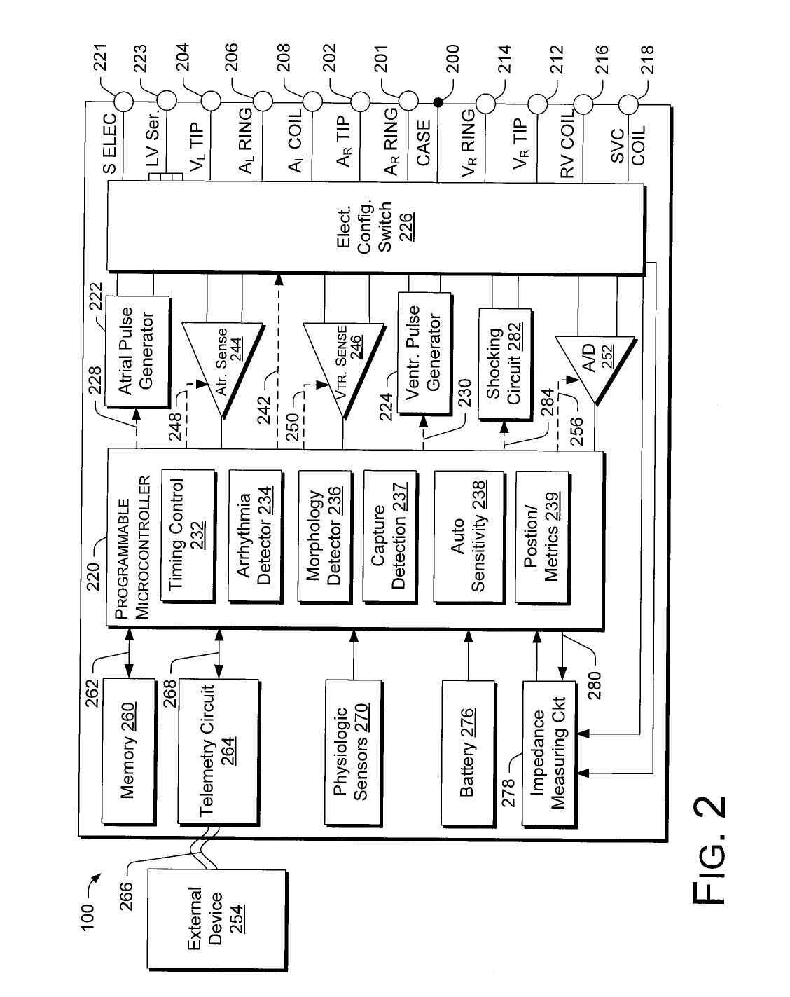

FIG. 2 shows an exemplary, simplified block diagram depicting various components of the device 100. The device 100 can be capable of treating both fast and slow arrhythmias with stimulation therapy, including cardioversion, defibrillation, and pacing stimulation. While a particular multi-chamber device is shown, it is to be appreciated and understood that this is for illustration purposes only. Thus, the techniques, methods, etc., described below can be implemented in connection with any suitably configured or configurable device. Accordingly, one of skill in the art could readily duplicate, eliminate, or disable the appropriate circuitry in any desired combination to provide a device capable of treating the appropriate chamber(s) or regions of a patient's heart.

Housing 200 for the device 100 is often referred to as the “can”, “case” or “case electrode”, and may be programmably selected to act as the return electrode for all “unipolar” modes. As described below, various exemplary techniques implement unipolar sensing for data that may include indicia of functional conduction block in myocardial tissue. Housing 200 may further be used as a return electrode alone or in combination with one or more of the coil electrodes 126, 132 and 134 for shocking or other purposes. Housing 200 further includes a connector (not shown) having a plurality of terminals 201, 202, 204, 206, 208, 212, 214, 216, 218, 221, 223 (shown schematically and, for convenience, the names of the electrodes to which they are connected are shown next to the terminals).

To achieve right atrial sensing, pacing and/or other tissue sensing, stimulation, etc., the connector includes at least a right atrial tip terminal (A_{R} TIP) 202 adapted for connection to the right atrial tip electrode 120. A right atrial ring terminal (A_{R} RING) 201 is also shown, which is adapted for connection to the right atrial ring electrode 121. To achieve left chamber sensing, pacing, shocking, and/or other tissue sensing, stimulation, etc., the connector includes at least a left ventricular tip terminal (V_{L} TIP) 204, a left atrial ring terminal (A_{L} RING) 206, and a left atrial shocking terminal (A_{L} COIL) 208, which are adapted for connection to the left ventricular tip electrode 122, the left atrial ring electrode 124, and the left atrial coil electrode 126, respectively. Connection to suitable stimulation electrodes is also possible via these and/or other terminals (e.g., via a stimulation terminal S ELEC 221). The terminal S ELEC 221 may optionally be used for sensing. For example, electrodes of the lead 110 may connect to the device 100 at the terminal 221 or optionally at one or more other terminals.

A terminal 223 allows for connection of a series of left ventricular electrodes. For example, the series of four electrodes 123 of the lead 106 may connect to the device 100 via the terminal 223. The terminal 223 and an electrode configuration switch 226 allow for selection of one or more of the series of electrodes and hence electrode configuration. In the example of FIG. 2, the terminal 223 includes four branches to the switch 226 where each branch corresponds to one of the four electrodes 123.

To support right chamber sensing, pacing, shocking, and/or other tissue sensing, stimulation, etc., the connector further includes a right ventricular tip terminal (V_{R} TIP) 212, a right ventricular ring terminal (V_{R} RING) 214, a right ventricular shocking terminal (RV COIL) 216, and a superior vena cava shocking terminal (SVC COIL) 218, which are adapted for connection to the right ventricular tip electrode 128, right ventricular ring electrode 130, the RV coil electrode 132, and the SVC coil electrode 134, respectively.

At the core of the stimulation device 100 is a programmable microcontroller 220 that controls the various modes of cardiac or other therapy. As is well known in the art, microcontroller 220 typically includes a microprocessor, or equivalent control circuitry, designed specifically for controlling the delivery of stimulation therapy, and may further include RAM or ROM memory, logic and timing circuitry, state machine circuitry, and I/O circuitry. Typically, microcontroller 220 includes the ability to process or monitor input signals (data or information) as controlled by a program code stored in a designated block of memory. The type of microcontroller is not critical to the described implementations. Rather, any suitable microcontroller 220 may be used that is suitable to carry out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052, the state-machine of U.S. Pat. Nos. 4,712,555 and 4,944,298, all of which are incorporated by reference herein. For a more detailed description of the various timing intervals used within the stimulation device and their inter-relationship, see U.S. Pat. No. 4,788,980, also incorporated herein by reference.

FIG. 2 also shows an atrial pulse generator 222 and a ventricular pulse generator 224 that generate pacing stimulation pulses for delivery by the right atrial lead 104, the coronary sinus lead 106, and/or the right ventricular lead 108 via an electrode configuration switch 226. It is understood that in order to provide stimulation therapy in each of the four chambers of the heart (or to other tissue) the atrial and ventricular pulse generators, 222 and 224, may include dedicated, independent pulse generators, multiplexed pulse generators, or shared pulse generators. The pulse generators 222 and 224 are controlled by the microcontroller 220 via appropriate control signals 228 and 230, respectively, to trigger or inhibit the stimulation pulses.

The microcontroller 220 further includes timing control circuitry 232 to control the timing of the stimulation pulses (e.g., pacing rate, atrio-ventricular (AV) delay, interatrial conduction (AA) delay, or interventricular conduction (VV) delay, etc.) as well as to keep track of the timing of refractory periods, blanking intervals, noise detection windows, evoked response windows, alert intervals, marker channel timing, etc., which is well known in the art.

The microcontroller 220 further includes an arrhythmia detector 234. The detector 234 can be utilized by the stimulation device 100 for determining desirable times to administer various therapies. The detector 234 may be implemented in hardware as part of the microcontroller 220, or as software/firmware instructions programmed into the device and executed on the microcontroller 220 during certain modes of operation.

Microcontroller 220 further includes a morphology discrimination module 236, a capture detection module 237 and an auto sensing module 238. These modules are optionally used to implement various exemplary recognition algorithms and/or methods presented below. The aforementioned components may be implemented in hardware as part of the microcontroller 220, or as software/firmware instructions programmed into the device and executed on the microcontroller 220 during certain modes of operation. The capture detection module 237, as described herein, may aid in acquisition, analysis, etc., of information relating to IEGMs and, in particular, act to distinguish capture versus non-capture versus fusion.

The microcontroller 220 further includes an optional position and/or metrics module 239. The module 239 may be used for purposes of acquiring position information, for example, in conjunction with a device (internal or external) that may use body surface patches or other electrodes (internal or external). The microcontroller 220 may initiate one or more algorithms of the module 239 in response to a signal detected by various circuitry or information received via the telemetry circuit 264. Instructions of the module 239 may cause the device 100 to measure potentials using one or more electrode configurations where the potentials correspond to a potential field generated by current delivered to the body using, for example, surface patch electrodes. Such a module may help monitor electrode positions and cardiac mechanics in relationship to cardiac electrical activity and may help to optimize cardiac resynchronization therapy. The module 239 may include instructions for vector analyses, for example, based on locally acquired or transmitted position information. The module 239 may operate in conjunction with various other modules and/or circuits of the device 100 (e.g., the impedance measuring circuit 278, the switch 226, the A/D 252, etc.).

The electronic configuration switch 226 includes a plurality of switches for connecting the desired electrodes to the appropriate I/O circuits, thereby providing complete electrode programmability. Accordingly, switch 226, in response to a control signal 242 from the microcontroller 220, determines the polarity of the stimulation pulses (e.g., unipolar, bipolar, etc.) by selectively closing the appropriate combination of switches (not shown) as is known in the art.

Atrial sensing circuits 244 and ventricular sensing circuits 246 may also be selectively coupled to the right atrial lead 104, coronary sinus lead 106, and the right ventricular lead 108, through the switch 226 for detecting the presence of cardiac activity in each of the four chambers of the heart. Accordingly, the atrial and ventricular sensing circuits, 244 and 246, may include dedicated sense amplifiers, multiplexed amplifiers, or shared amplifiers. Switch 226 determines the “sensing polarity” of the cardiac signal by selectively closing the appropriate switches, as is also known in the art. In this way, the clinician may program the sensing polarity independent of the stimulation polarity. The sensing circuits (e.g., 244 and 246) are optionally capable of obtaining information indicative of tissue capture.

Each of the sensing circuits 244 and 246 preferably employs one or more low power, precision amplifiers with programmable gain and/or automatic gain control, bandpass filtering, and a threshold detection circuit, as known in the art, to selectively sense the cardiac signal of interest. The automatic gain control enables the device 100 to deal effectively with the difficult problem of sensing the low amplitude signal characteristics of atrial or ventricular fibrillation.

The outputs of the atrial and ventricular sensing circuits 244 and 246 are connected to the microcontroller 220, which, in turn, is able to trigger or inhibit the atrial and ventricular pulse generators 222 and 224, respectively, in a demand fashion in response to the absence or presence of cardiac activity in the appropriate chambers of the heart. Furthermore, as described herein, the microcontroller 220 is also capable of analyzing information output from the sensing circuits 244 and 246 and/or the data acquisition system 252 to determine or detect whether and to what degree tissue capture has occurred and to program a pulse, or pulses, in response to such determinations. The sensing circuits 244 and 246, in turn, receive control signals over signal lines 248 and 250 from the microcontroller 220 for purposes of controlling the gain, threshold, polarization charge removal circuitry (not shown), and the timing of any blocking circuitry (not shown) coupled to the inputs of the sensing circuits, 244 and 246, as is known in the art.

For arrhythmia detection, the device 100 may utilize the atrial and ventricular sensing circuits, 244 and 246, to sense cardiac signals to determine whether a rhythm is physiologic or pathologic. Of course, other sensing circuits may be available depending on need and/or desire. In reference to arrhythmias, as used herein, “sensing” is reserved for the noting of an electrical signal or obtaining data (information), and “detection” is the processing (analysis) of these sensed signals and noting the presence of an arrhythmia or of a precursor or other factor that may indicate a risk of or likelihood of an imminent onset of an arrhythmia.

The exemplary detector module 234, optionally uses timing intervals between sensed events (e.g., P-waves, R-waves, and depolarization signals associated with fibrillation) and to perform one or more comparisons to a predefined rate zone limit (i.e., bradycardia, normal, low rate VT, high rate VT, and fibrillation rate zones) and/or various other characteristics (e.g., sudden onset, stability, physiologic sensors, and morphology, etc.) in order to determine the type of remedial therapy (e.g., anti-arrhythmia, etc.) that is desired or needed (e.g., bradycardia pacing, anti-tachycardia pacing, cardioversion shocks or defibrillation shocks, collectively referred to as “tiered therapy”). Similar rules can be applied to the atrial channel to determine if there is an atrial tachyarrhythmia or atrial fibrillation with appropriate classification and intervention.

Cardiac signals are also applied to inputs of an analog-to-digital (A/D) data acquisition system 252. The data acquisition system 252 is configured to acquire intracardiac electrogram (IEGM) signals or other action potential signals, convert the raw analog data into a digital signal, and store the digital signals for later processing and/or telemetric transmission to an external device 254. The data acquisition system 252 is coupled to the right atrial lead 104, the coronary sinus lead 106, the right ventricular lead 108 and/or another lead (e.g., the lead 110) through the switch 226 to sample cardiac signals or other signals across any pair or other number of desired electrodes. A control signal 256 from the microcontroller 220 may instruct the A/D 252 to operate in a particular mode (e.g., resolution, amplification, etc.).

Various exemplary mechanisms for signal acquisition are described herein that optionally include use of one or more analog-to-digital converter. Various exemplary mechanisms allow for adjustment of one or more parameter associated with signal acquisition.

The microcontroller 220 is further coupled to a memory 260 by a suitable data/address bus 262, wherein the programmable operating parameters used by the microcontroller 220 are stored and modified, as required, in order to customize the operation of the stimulation device 100 to suit the needs of a particular patient. Such operating parameters define, for example, pacing pulse amplitude, pulse duration, electrode polarity, rate, sensitivity, automatic features, arrhythmia detection criteria, and the amplitude, waveshape, number of pulses, and vector of each shocking pulse to be delivered to the patient's heart 102 within each respective tier of therapy. One feature of the described embodiments is the ability to sense and store a relatively large amount of data (e.g., from the data acquisition system 252), which data may then be used for subsequent analysis to guide the programming and operation of the device 100.

Advantageously, the operating parameters of the implantable device 100 may be non-invasively programmed into the memory 260 through a telemetry circuit 264 in telemetric communication via communication link 266 with the external device 254, such as a programmer, transtelephonic transceiver, or a diagnostic system analyzer. The microcontroller 220 activates the telemetry circuit 264 with a control signal 268. The telemetry circuit 264 advantageously allows intracardiac electrograms (IEGM) and other information (e.g., status information relating to the operation of the device 100, etc., as contained in the microcontroller 220 or memory 260) to be sent to the external device 254 through an established communication link 266.

The stimulation device 100 can further include one or more physiologic sensors 270. For example, the device 100 may include a “rate-responsive” sensor that may provide, for example, information to aid in adjustment of pacing stimulation rate according to the exercise state of the patient. However, the one or more physiological sensors 270 may further be used to detect changes in cardiac output (see, e.g., U.S. Pat. No. 6,314,323, entitled “Heart stimulator determining cardiac output, by measuring the systolic pressure, for controlling the stimulation”, to Ekwall, issued Nov. 6, 2001, which discusses a pressure sensor adapted to sense pressure in a right ventricle and to generate an electrical pressure signal corresponding to the sensed pressure, an integrator supplied with the pressure signal which integrates the pressure signal between a start time and a stop time to produce an integration result that corresponds to cardiac output), changes in the physiological condition of the heart, or diurnal changes in activity (e.g., detecting sleep and wake states). Accordingly, the microcontroller 220 responds by adjusting the various pacing parameters (such as rate, AV Delay, VV Delay, etc.) at which the atrial and ventricular pulse generators, 222 and 224, generate stimulation pulses.

While shown as being included within the stimulation device 100, it is to be understood that one or more of the physiologic sensors 270 may also be external to the stimulation device 100, yet still be implanted within or carried by the patient. Examples of physiologic sensors that may be implemented in device 100 include known sensors that, for example, sense respiration rate, oxygen concentration of blood, pH of blood, CO₂ concentration of blood, ventricular gradient, cardiac output, preload, afterload, contractility, and so forth. Another sensor that may be used is one that detects activity variance, wherein an activity sensor is monitored diurnally to detect the low variance in the measurement corresponding to the sleep state. For a complete description of the activity variance sensor, the reader is directed to U.S. Pat. No. 5,476,483 which is hereby incorporated by reference.

The one or more physiologic sensors 270 optionally include sensors for detecting movement and minute ventilation in the patient. Signals generated by a position sensor, a MV sensor, etc., may be passed to the microcontroller 220 for analysis in determining whether to adjust the pacing rate, etc. The microcontroller 220 may monitor the signals for indications of the patient's position and activity status, such as whether the patient is climbing upstairs or descending downstairs or whether the patient is sitting up after lying down.

The stimulation device 100 additionally includes a battery 276 that provides operating power to all of the circuits shown in FIG. 2. For the stimulation device 100, which employs shocking therapy, the battery 276 is capable of operating at low current drains for long periods of time (e.g., preferably less than 10 μA), and is capable of providing high-current pulses (for capacitor charging) when the patient requires a shock pulse (e.g., preferably, in excess of 2 A, at voltages above 200 V, for periods of 10 seconds or more). The battery 276 also desirably has a predictable discharge characteristic so that elective replacement time can be detected.

The stimulation device 100 can further include magnet detection circuitry (not shown), coupled to the microcontroller 220, to detect when a magnet is placed over the stimulation device 100. A magnet may be used by a clinician to perform various test functions of the stimulation device 100 and/or to signal the microcontroller 220 that the external programmer 254 is in place to receive or transmit data to the microcontroller 220 through the telemetry circuits 264.

The stimulation device 100 further includes an impedance measuring circuit 278 that is enabled by the microcontroller 220 via a control signal 280. The known uses for an impedance measuring circuit 278 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 278 is advantageously coupled to the switch 226 so that any desired electrode may be used.

In the case where the stimulation device 100 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 220 further controls a shocking circuit 282 by way of a control signal 284. The shocking circuit 282 generates shocking pulses of low (e.g., up to 0.5 J), moderate (e.g., 0.5 J to 10 J), or high energy (e.g., 11 J to 40 J), as controlled by the microcontroller 220. Such shocking pulses are applied to the patient's heart 102 through at least two shocking electrodes, and as shown in this embodiment, selected from the left atrial coil electrode 126, the RV coil electrode 132, and/or the SVC coil electrode 134. As noted above, the housing 200 may act as an active electrode in combination with the RV electrode 132, or as part of a split electrical vector using the SVC coil electrode 134 or the left atrial coil electrode 126 (i.e., using the RV electrode as a common electrode).

Cardioversion level shocks are generally considered to be of low to moderate energy level (so as to minimize pain felt by the patient), and/or synchronized with an R-wave and/or pertaining to the treatment of tachycardia. Defibrillation shocks are generally of moderate to high energy level (e.g., corresponding to thresholds in the range of approximately 5 J to 40 J), delivered asynchronously (since R-waves may be too disorganized), and pertaining exclusively to the treatment of fibrillation. Accordingly, the microcontroller 220 is capable of controlling the synchronous or asynchronous delivery of the shocking pulses.

As already mentioned, the implantable device 100 includes impedance measurement circuitry 278. Such a circuit may measure impedance or electrical resistance through use of various techniques. For example, the device 100 may deliver a low voltage (e.g., about 10 mV to about 20 mV) of alternating current between the RV tip electrode 128 and the case electrode 200. During delivery of this energy, the device 100 may measure resistance between these two electrodes where the resistance depends on any of a variety of factors. For example, the resistance may vary inversely with respect to volume of blood along the path.

In another example, resistance measurement occurs through use of a four terminal or electrode technique. For example, the exemplary device 100 may deliver an alternating current between one of the RV tip electrode 128 and the case electrode 200. During delivery, the device 100 may measure a potential between the RA ring electrode 121 and the RV ring electrode 130 where the potential is proportional to the resistance between the selected potential measurement electrodes.

With respect to two terminal or electrode techniques, where two electrodes are used to introduce current and the same two electrodes are used to measure potential, parasitic electrode-electrolyte impedances can introduce noise, especially at low current frequencies; thus, a greater number of terminals or electrodes may be used. For example, aforementioned four electrode techniques, where one electrode pair introduces current and another electrode pair measures potential, can cancel noise due to electrode-electrolyte interface impedance. Alternatively, where suitable or desirable, a two terminal or electrode technique may use larger electrode areas (e.g., even exceeding about 1 cm²) and/or higher current frequencies (e.g., above about 10 kHz) to reduce noise.

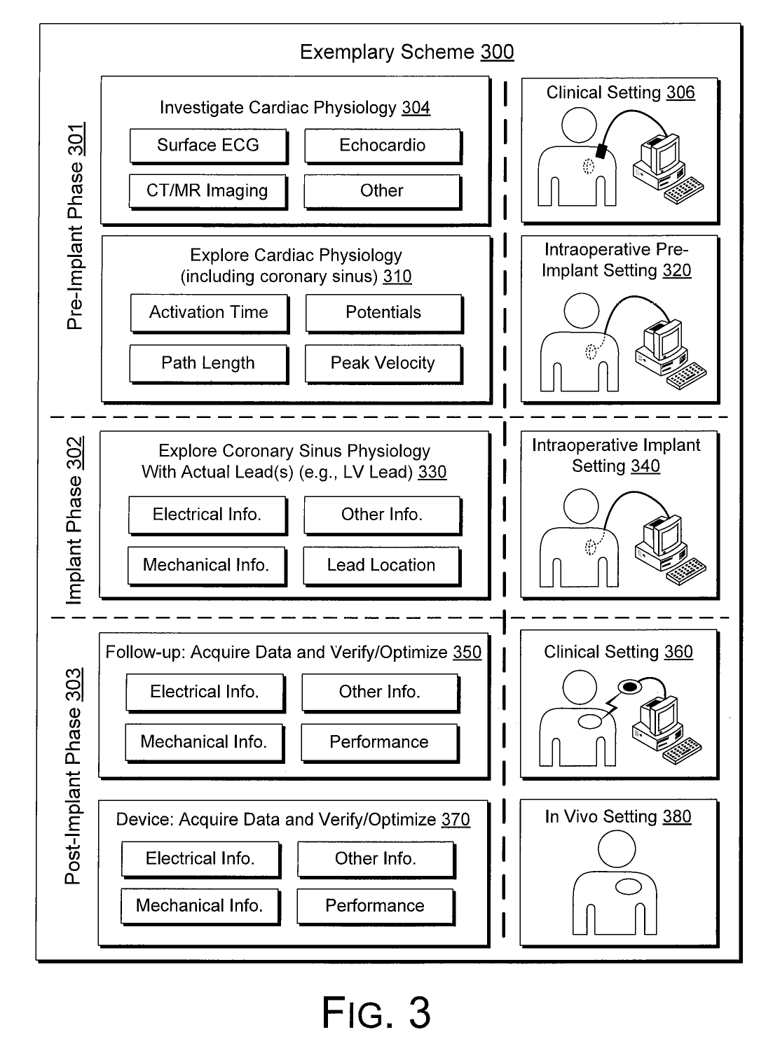

FIG. 3 shows an exemplary scheme 300 that spans a pre-implant phase 301, an implant phase 302 and a post-implant phase 303. In the example of FIG. 3, the pre-implant phase 301 is shown as including a clinical setting 306 and an intraoperative (acute) pre-implant setting 320; the implant phase 302 as including an intraoperative (acute) implant setting 340; and the post-implant phase 303 as including a clinical (chronic) setting 360 and an in vivo (chronic) setting 380. The scheme 300 provides for acquisition and analysis of pre-implant phase 301 information to enhance the implant phase 302 and optionally the post-implant phase 303.

In an investigation block 304 taking place in the clinical setting 306, various types of information may be acquired to understand better the patient's cardiac physiology and performance. For example, a clinician may acquire a surface ECG, an echocardiogram, images of cardiac physiology, etc., and analyze the acquired information as part of a diagnostic process or a pre-implant treatment planning process. A diagnostic or treatment planning processes may rely on electrical information (e.g., ECG), mechanical information (e.g., echo, CT, MRI, etc.) or a combination of electrical and mechanical information. In the clinical setting 306, electrical and mechanical information may be acquired simultaneous. For example, consider acquisition of CT, MR or echo data using ECG gating, which can help to determine performance metrics such as cardiac output, chamber volume, blood flow velocities, etc., at one or more times during a cardiac cycle (e.g., peak systolic, peak diastolic, etc.).

A clinician may make any of a variety of recommendations based on the clinical investigation 304. Where a recommendation includes one or more surgical procedures, an opportunity exists to acquire additional data. Where successive surgical procedures are recommended, data acquired from one surgical procedure can assist in performance of a subsequent surgical procedure. As described herein, an exemplary process of data acquisition and analysis can expedite a surgical procedure, increase treatment efficacy or both.

Referring to the intraoperative pre-implant setting 320 of FIG. 3, exploration of cardiac performance and physiology 310 may occur in this setting. Such an exploration allows for acquisition of various types of information in the intraoperative setting 320. Such exploration may occur as part of cardiac ablation surgery, cardiac arterial bypass surgery, cardiac stent surgery or cardiac valve repair or replacement surgery or, alternatively, merely for purposes of pre-implant exploration.

As described herein, the exploration procedure 310 relies on a localization system such as the ENSITE® NAVX® system or other system with appropriate localization features. The ENSITE® NAVX® localization system includes patch electrodes for placement on a patient's body that can establish a multidimensional localization field (e.g., by delivery of current using patch electrodes). Given a localization field, the ENSITE® NAVX® system can use an electrode positioned in the body of the patient to measure electrical potential and, in turn, to determine a position for the electrode. Where an electrode is positioned in a cardiac space (e.g., cardiac surface, cardiac chamber, cardiac vein, etc.), the ENSITE® NAVX® system can acquire electrical potential with respect to time to generate a mechanical waveform indicative of cardiac motion. Such a waveform may be analyzed (or acquired) with respect to electrical information, for example, to determine position, displacement, velocity, acceleration, etc., of an electrode in response to cardiac motion (e.g., peak systolic, peak diastolic, etc.).

As shown in the exploration block 310 of FIG. 3, electrical information such as activation times and cardiac potentials and mechanical information such as path length and peak velocity may be acquired or determined during a pre-implant phase. As described in more detail below, such information may be acquired or determined with respect to a venous network of the heart. The primary venous network of the heart includes the coronary sinus, which empties into the right atrium via the coronary sinus ostium. The coronary sinus network drains about 95% of the venous blood of the myocardium (remaining 5% of myocardial venous flow drains through the thebesian vessels).

The coronary sinus has various tributary veins including the small, middle, great and oblique cardiac veins, the left marginal vein and the left posterior ventricular vein. The great cardiac vein is normally the longest venous vessel of the heart. The great and the middle cardiac veins normally merge at the apex of the heart, forming together with the coronary sinus, a fairly complete venous ring around the left ventricle. Consequently, these tributaries of the coronary sinus are often considered when deciding where to place a lead for electrical activation of the left ventricle.

Intraoperative exploration performed per the exploration block 310 depends on catheter characteristics. For example, a catheter with a large cross-sectional dimension or high rigidity may be suited for navigation of the coronary sinus but only partial navigation of one or more tributaries of the coronary sinus. In contrast, leads typically configured for stimulation therapies have small cross-section dimension and are quite flexible to allow for deep access to the heart's venous network.

In some instances, a catheter may be configured to acquire data such as temperature or flow (e.g., thermodilution). In such instances, flow, temperature or other data may be acquired during the intraoperative exploration 310. While blood from the coronary sinus drains to the heart, flow to the coronary sinus still effectively transports heat energy to aid in cooling the heart. Various studies demonstrate relationships between flow in the coronary sinus or tributaries thereof with conditions such as ischemia. Such information may help localize ischemia and, as described herein, improve selection of an appropriate venous branch for locating one or more lead-based electrodes. Where such information is localized using a localization system, the information may be mapped or otherwise presented or analyzed in conjunction with localized electrical information, mechanical information, etc. Accordingly, a rich understanding of a patient's venous network, particularly the coronary sinus, may be attained prior to actual implantation of an implantable cardiac therapy device.

As described herein, an exemplary method includes selecting one or more tributaries of the coronary sinus as a candidate (or candidates) for lead placement based at least in part on information acquired during an intraoperative procedure. As shown in the exemplary scheme 300 of FIG. 3, lead placement occurs in a subsequent procedure 330, in an intraoperative setting 340, for implantation of an implantable cardiac therapy device.

In the example of FIG. 3, the exemplary scheme 300 includes the intraoperative exploration procedure 330 where exploration occurs using one or more implantable leads. As mentioned, an implantable lead, depending on its characteristics, may be able to navigate a venous network more thoroughly than a catheter (e.g., due to smaller cross-sectional dimension, flexibility, etc.). However, a full exploration of the venous network may take considerable time. Therefore, an exemplary implant planning process includes selecting less than all of the tributaries to the coronary sinus for exploration. As described herein, such a planning process relies, at least in part, on information acquired during a prior interoperative procedure. For example, a catheter may be well suited to explore the coronary sinus, especially regions of confluence with its tributaries. Based on such an exploration, a particular tributary may be selected for implant of a lead where, upon implantation of the lead, a subsequent exploration identifies an optimal location in the selected tributary.

As indicated in FIG. 3, the exploration procedure 330 may acquire electrical information, mechanical information and other information to determine one or more locations for chronic placement of a lead. The procedure 330 may rely on a localization system such as the ENSITE® NAVX® system to acquire position information. As mentioned, position information may be used to determine local motion, velocity and acceleration and may be combined with electrical information to provide local electrical-mechanical delays and the like. A localization system may include mapping features that allow for essentially real-time display of mapped information as such information is acquired during an exploration of the venous network of a patient. As described herein, real-time information may be mapped in conjunction with previously acquired information from a prior intraoperative procedure and optionally other information (e.g., image information from CT, MR or ultrasound studies).

During the procedure 330, a clinician may explore a venous network while delivering electrical energy to stimulate the heart. Further, delivery parameters may be varied to determine whether a location in a selected tributary of the coronary sinus is suitable for chronic pacing or stimulation therapy. For example, a clinician may vary polarity, energy level, pulse shape, pulse duration, etc., during the procedure 330 while acquiring position information (e.g., electrical potentials measured in a localization field). Where the procedure 330 inserts multiple electrode-bearing leads, various electrodes on those leads may be used to acquire position information, for example, to understand cardiac mechanics responsive to the delivered stimulation energy. Further, such electrodes may acquire potentials associated with cardiac activity. Accordingly, a mapping process may map mechanical and electrical information to a display in near real-time to allow a clinician to expeditiously explore a tributary to the coronary sinus and select an optimal location for one or more lead-based electrodes.

In the post-implant or chronic phase 303, a follow-up procedure 350 typically takes place in a clinical setting 360 to acquire data and verify or optimize parameters associated with the implanted cardiac therapy device. Depending on the capabilities of the device and clinical equipment, various types of information may be acquired. As explained with respect to the device 100 of FIGS. 1 and 2, a typical device is configured for telemetric communication with an external device, sometimes referred to as a device programmer. The device may transmit acquired information to an external device and respond to instructions received from an external device. An implanted device may transmit IEGMs (electrical information) as well as other information (e.g., depending of device capabilities). For example, with respect to mechanical information, the implanted device may include an accelerometer, impedance circuitry, etc., which may be used to acquire information related to cardiac mechanics. An implanted device or an external device may assess cardiac performance based on acquired information. In turn, one or more therapy parameters may be verified or optimized. Further, depending on the clinical setting 360, echocardiography, CT or other equipment may be available to acquire information to aid in an assessment of cardiac performance, implanted device performance, etc. Yet further, an external system may be available to generate a localization field where implanted electrodes can measure electrical potential in the localization field. Where such a system is available, the follow-up procedure 350 may include verification or optimization based on such position information (e.g., akin to the aforementioned ENSITE® NAVX® system analyses).

As described herein, where an exemplary coronary sinus mapping technique is used to enhance CRT, one may expect values for time from RV pace to electrical activation of the LV to become more homogeneous after commencement of CRT therapy. Further, a coronary sinus map may be used as an indicator of potential CRT efficacy or response and optionally, after delivery of CRT, to determine whether a patient is a CRT responder. In addition, where electrode position can be determined post-implant, lead motion data may be compared to baseline measurements taken at the time of coronary sinus mapping or CRT implant (or both).

After implantation and between follow-up visits, a device-based acquisition process 370 may acquire various types of information including electrical information and optionally mechanical information. An implanted device may be configured to acquire information and to verify or optimize one or more parameters based on such information. For example, the QUICKOPT® algorithm (St. Jude Medical Cardiac Rhythm Management Division) can allow for device-based verification or optimization of AV and VV delays based on acquired electrical information.

As described herein, data acquired during the pre-implant phase 301, the implant phase 302 and the post-implant phase 303, or analyses based on such data, may be stored in a database. Where a database stores data or analyses for many patients, it may be relied on during any of the various phases of the scheme 300 of FIG. 3. Information may be used to track progress of a patient over time. Further, a trend for a patient or implanted device may be compared to trends for other patients or other implanted devices. As described in more detail below, one or more indexes may be used to assist in locating a lead or electrode. Depending on capabilities, such indexes may be tracked over time for a patient or patients. As to storage, information may be stored in an implantable device, a programmer configured with storage, a networked storage device, a removable storage device (e.g., a memory card), etc. Where an implantable device stores data acquired during one or more phases, the data may be relied on in making decisions as to delivery of therapy (e.g., setting one or more therapy parameters, trend analysis, etc.).

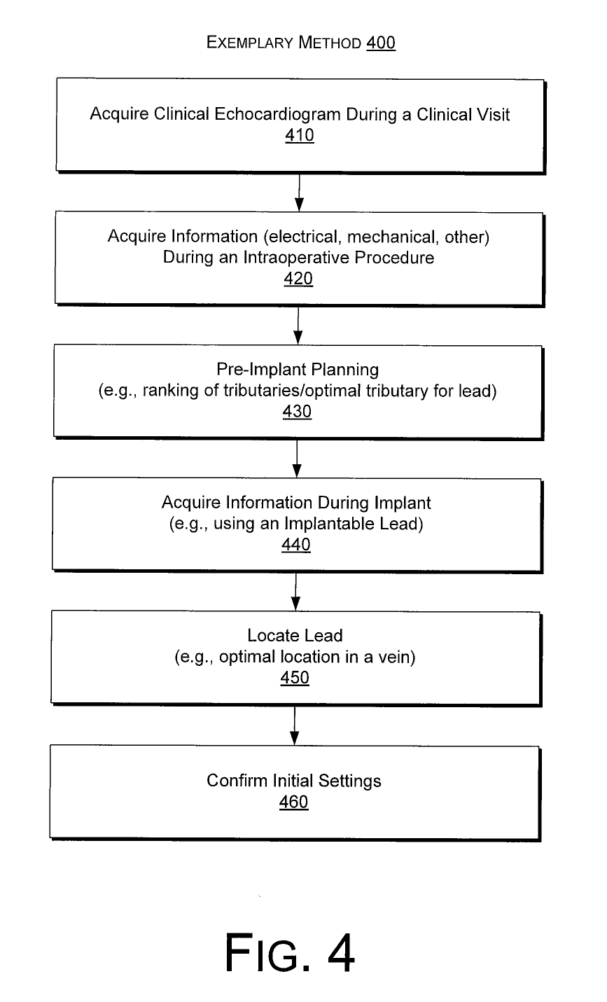

FIG. 4 shows an exemplary method 400 that spans pre-implant and implant phases. The method 400 commences in an acquisition block 410 where a clinician acquires an echocardiogram during a clinical visit. In a subsequent acquisition block 420, a clinician acquires information during an intraoperative procedure where a catheter is positioned in the venous network of a patient. Specifically, the procedure involves positioning the catheter in the coronary sinus of a patient to acquire information at various locations in the coronary sinus, especially in regions of confluence (e.g., where a tributary vein joins the coronary sinus). In the example of FIG. 4, a pre-implant planning procedure 430 follows that aims to select a particular tributary for placement of an implantable lead. In essence, the pre-implant planning procedure 430 relies on the previously acquired information to decide which tributary is optimal for placement of an implantable lead for delivery of a cardiac therapy. As described in more detail below, such a pre-implant planning procedure may rely on a venous network map that map one or more measures or metrics in association with the coronary sinus. Such a visual presentation of the coronary sinus (e.g., in three-dimensions) can facilitate selection of a tributary for lead placement, especially for cardiac therapies that include left ventricular stimulation (e.g., CRT).

After selection of a particular tributary, in an acquisition block 440, acquisition of information occurs during exploration of the selected tributary using an implantable lead. In the example of FIG. 4, the acquisition procedure 440 relies on a localization system to acquire position information. Such position information may be used to understand cardiac electrical activity and mechanics (intrinsic or responsive to stimulation), which, in turn, can help optimize lead or electrode location in the selected tributary of the coronary sinus.

After the exploration block 440, in a location block 450, a clinician locates a lead in the selected tributary (e.g., typically the lead used for exploration). Once located, in a confirmation block 460, a clinician may verify initial settings for delivery of a cardiac therapy that relies on the implanted lead.

The method 400 can save a clinician considerable time during an implant procedure. Specifically, where a clinician knows a priori which branch of the coronary sinus to locate a lead, through use of a localization system (and optionally fluoroscopy), the clinician can readily locate the lead. Further, the clinician can quickly explore various locations in the branch to optimize the location of the lead. A visual presentation of the coronary sinus and its tributaries can also help familiarize a clinician with a patient's anatomy, as anatomy of the cardiac venous network tends to differ somewhat from person to person. In essence, the clinician does not need to explore the coronary sinus during implant but only a select tributary thereof. Such a process can reduce risk of damage to cardiac veins as at least some of the anatomy is known a priori and as not every tributary need be explored with a lead.

In a variation of the method 400, a clinician may simply position a lead in the selected tributary by a pre-determined distance from a point of confluence. For example, a planning procedure may recommend placement of a lead 3 cm from a point of confluence. During implantation, the clinician can locate the tip of the lead (distal end) at the point of confluence and then insert the lead 3 cm into the tributary. As described herein, various levels of optimization may be performed depending on circumstances (e.g., patient condition, type of therapy, etc.). For example, where a patient is indicated as a borderline responder to CRT, a clinician may take additional time to optimize location of a lead in a tributary of the coronary sinus. Such an optimization aims to increase the likelihood that the patient will respond to the CRT.

As described herein, an exemplary method includes intraoperative pre-implantation (e.g., intraoperative catheterization) and implantation procedures (e.g., intraoperative CRT implantation) to optimize target vein selection and intrabranch site selection. Such a method accounts for both mechanical and electrical activation patterns of the heart, for example, according to data acquired using a localization system (e.g., ENSITE® NAVX® system). Information acquired during such an exemplary method can help assess therapy (e.g., CRT) efficacy during follow-up visits, for example, by comparing electrical activation times to those acquired during exploration of the venous network.

An exemplary method may include preparing a patient for a pre-implant electroanatomic mapping study. Such preparation may occur in a relatively standard manner for using the ENSITE® NAVX® system or other similar technology. As described herein, any of a variety of electroanatomic mapping or locating systems that can locate indwelling electrodes in and around the heart may be used. Once prepped, a clinician or robot may place leads and/or catheters in the patient's body to acquire information about venous structure, especially the coronary sinus and regions of confluence with at least some of its tributaries.

After such a mapping study, the exemplary method may include preparing the patient for both implant of a device (such as the device 100 of FIGS. 1 and 2) and for an electroanatomic mapping study. Once prepped, a clinician or robot may place leads and/or catheters in the patient's body, including any leads to be chronically implanted as part of a therapy system (e.g., CRT), as well as optional additional electrodes that may yield additional information (e.g., to increase accuracy by providing global information or other information).

In either the pre-implant or implant procedures, after an initial placement of an electrode-bearing catheter or an electrode-bearing lead, a clinician may connect one or more electrodes to an electroanatomic mapping or localization system. The term “connection” can refer to physical electrical connection or wireless connection (e.g., telemetric, RF, ultrasound, etc.) with the electrodes or wireless connection with another device that is in electrical contact with the electrodes.

Once an appropriate connection or connections have been made, real-time position data for one or more electrodes may be acquired for various configurations or conditions. For example, position data may be acquired during normal sinus rhythm; pacing in one or more chambers; advancing, withdrawing, or moving a location of an electrode; pacing one or more different electrode configurations (e.g. multisite pacing); or varying inter-stimulus timing (e.g. AV delay, VV delay).

In various examples, simultaneous to the position recording, an intracardiac electrogram (IEGM) from each electrode can also be recorded and associated with the anatomic position of the electrode. While various examples refer to simultaneous acquisition, acquisition of electrical information and acquisition of position information may occur sequentially (e.g., alternate cardiac cycles) or interleaved (e.g., both acquired during the same cardiac cycle but offset by sampling time or sampling frequency).

In various exemplary methods, electrodes within the cardiac space may be optionally positioned at various locations (e.g., by continuous movement or by discrete, sequential moves), with a localization system recording the real-time position information at each electrode position in a point-by-point manner. Such position data can by associated with a respective anatomic point from which it was collected. By moving the electrodes from point to point during an intervention, the position data from each location can be analyzed, optionally to provide one or more metrics.

As explained, an exemplary method can include mapping one or more metrics, optionally in conjunction with one or more configuration parameters. In turn, an algorithm or a clinician may select a configuration (e.g., electrode location, multisite arrangement, AV/VV timing, pacing voltage, etc.) that yielded the best value for cardiac performance and use the selected configuration as a chronic configuration for the CRT system. Such a chronic configuration may be optionally updated from time to time (e.g., during a follow-up visit, in a patient environment, etc., depending on specific capabilities of a system).

Various exemplary methods, using either a single metric or a combination of more than one metric, may automatically select a configuration, present an optimal configuration for acknowledgement by a clinician, or present various configurations to a clinician along with pros and cons of each configuration (e.g., in objective or subjective terms). Pros and cons may pertain to cardiac performance, patient comfort (e.g., pain, lack of pain, overall feeling, etc.), device performance, etc. As described herein, various decisions are based on one or more vector metrics.

An exemplary method may rely on certain equipment at time of implant or exploration and other equipment after implantation of a device to deliver a cardiac therapy. For example, during an intraoperative procedure, wireless communication may not be required; whereas, during a follow-up visit, measured potentials for position of chronically implanted electrodes (e.g., mechanical information) and of measured IEGMs using chronically implanted electrodes (e.g., electrical information) may be communicated wirelessly from an implanted device to an external device. With respect to optimization or assessment of a chronically implanted system, in general, electrode location will not be altered (e.g., except for dislocation or failure), but other parameters altered to result in an optimal configuration (e.g., single- or multi-site arrangement, polarity, stimulation energy, timing parameters, etc.).

As discussed herein, various exemplary techniques deliver current and measure potential where potential varies typically with respect to cardiac mechanics (e.g., due to motion). For example, electrodes for delivery of current may be placed at locations that do not vary significantly with respect to cardiac mechanics or other patient motion (e.g., breathing) while one or more electrodes for measuring potential may be placed at a location or locations that vary with respect to cardiac mechanics or other patient motion. Alternatively, electrodes for measuring potential may be placed at locations that do not vary significantly with respect to cardiac mechanics or other patient motion while one or more electrodes for delivery of current may be placed at a location or locations that vary with respect to cardiac mechanics or other patient motion. Various combinations of the foregoing arrangements are possible as well. Electrodes may be associated with a catheter or a lead. In some instances, an electrode may be a “stand-alone” electrode, such as a case electrode of an implantable device (see, e.g., the case electrode 200 of the device 100 of FIGS. 1 and 2).

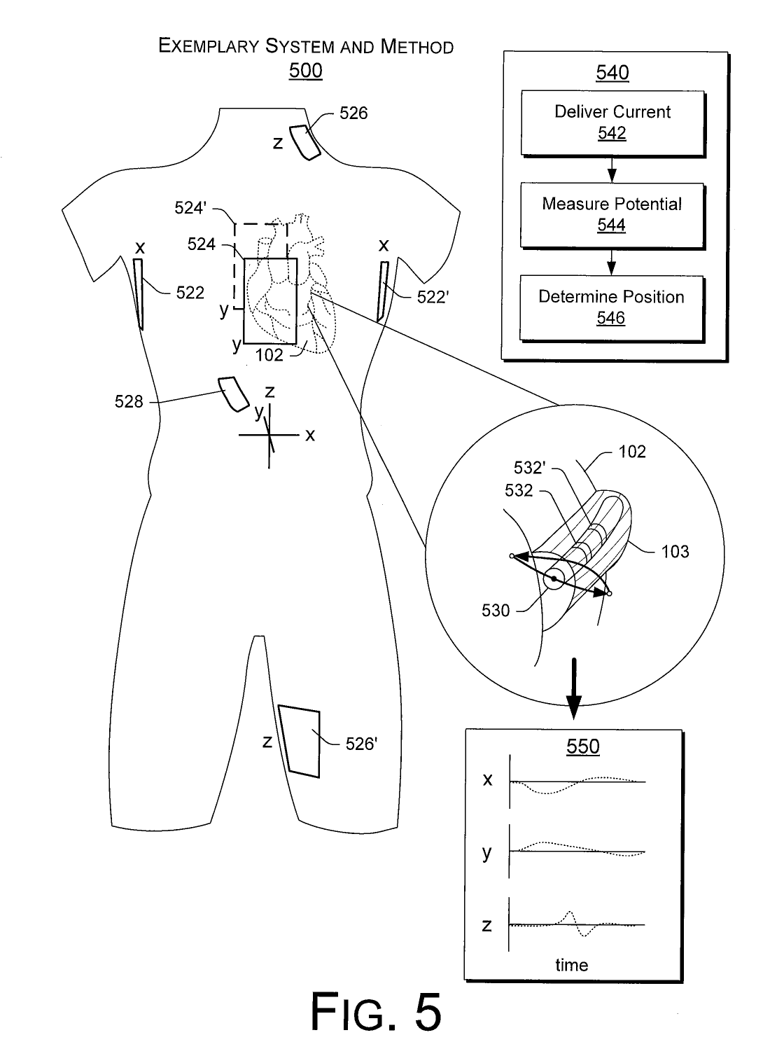

FIG. 5 shows an arrangement and method 500 that may rely in part on a commercially available system marketed as ENSITE® NAVX® navigation and visualization system (see also LOCALISM) system, Medtronic, Inc., Minnesota). The ENSITE® NAVX® system is a computerized storage and display system for use in electrophysiology studies of the human heart. The system consists of a console workstation, patient interface unit, and an electrophysiology mapping catheter and/or surface electrode kit. By visualizing the global activation pattern seen on color-coded isopotential maps in the system, in conjunction with the reconstructed electrograms, an electrophysiologist can identify the source of an arrhythmia and can navigate to a defined area for therapy. The ENSITE® system is also useful in treating patients with simpler arrhythmias by providing non-fluoroscopic navigation and visualization of conventional electrophysiology (EP) catheters.

As shown in FIG. 5, electrodes 532, 532′, which may be part of a standard EP catheter 530 (or lead), sense electrical potential associated with current signals transmitted between three pairs of surface electrode patches 522, 522′ (x-axis), 524, 524′ (y-axis) and 526, 526′ (z-axis). An addition electrode patch 528 (sometimes referred to as a “belly” patch) is available for reference, grounding or other function. The ENSITE® NAVX® system can also collect electrical data from a catheter and can plot a cardiac electrogram from a particular location (e.g., cardiac vein 103 of heart 102). Information acquired may be displayed as a 3-D isopotential map and as virtual electrograms. Repositioning of the catheter allows for plotting of cardiac electrograms from other locations. Multiple catheters may be used as well. A cardiac electrogram or electrocardiogram (ECG) of normal heart activity (e.g., polarization, depolarization, etc.) typically shows atrial depolarization as a “P wave”, ventricular depolarization as an “R wave”, or QRS complex, and repolarization as a “T wave”. The ENSITE® NAVX® system may use electrical information to track or navigate movement and construct three-dimensional (3-D) models of a chamber of the heart.

A clinician can use the ENSITE® NAVX® system to create a 3-D model of a chamber in the heart for purposes of treating arrhythmia (e.g., treatment via tissue ablation). To create the 3-D model, the clinician applies surface patches to the body. The ENSITE® NAVX® system transmits an electrical signal between the patches and the system then senses the electrical signal using one or more catheters positioned in the body. The clinician may sweep a catheter with electrodes across a chamber of the heart to outline structure. Signals acquired during the sweep, associated with various positions, can then be used to generate a 3-D model. A display can display a diagram of heart morphology, which, in turn, may help guide an ablation catheter to a point for tissue ablation.

With respect to the foregoing discussion of current delivery and potential measurement, per a method 540, a system (e.g., such as the ENSITE® NAVX® system) delivers low level separable currents from the three substantially orthogonal electrode pairs (522, 522′, 524, 524′, 526, 526′) positioned on the body surface (delivery block 542). The specific position of a catheter (or lead) electrode within a chamber of the heart can then be established based on three resulting potentials measured between the recording electrode with respect to a reference electrode, as seen over the distance from each patch set to the recording tip electrode (measurement block 544). Sequential positioning of a catheter (or lead) at multiple sites along the endocardial surface of a specific chamber can establish that chamber's geometry, i.e., position mapping (position/motion determination block 546). Where the catheter (or lead) 530 moves, the method 540 may also measure motion.

In addition to mapping at specific points, the ENSITE® NAVX® system provides for interpolation (mapping a smooth surface) onto which activation voltages and times can be registered. Around 50 points are required to establish a surface geometry and activation of a chamber at an appropriate resolution. The ENSITE® NAVX® system also permits the simultaneous display of multiple catheter electrode sites, and also reflects real-time motion of both ablation catheters and those positioned elsewhere in the heart.

The ENSITE® NAVX® system relies on catheters for temporary placement in the body. Various exemplary techniques described herein optionally use one or more electrodes for chronic implantation. Such electrodes may be associated with a lead, an implantable device, or other chronically implantable component.

With respect to motion (e.g., change in position with respect to time), the exemplary system and method 500 may track motion of an electrode in one or more dimensions. For example, a plot 550 of motion versus time for three dimensions corresponds to motion of one or more electrodes of the catheter (or lead) 530 positioned in a vessel 103 of the heart 102 where the catheter (or lead) 530 includes the one or more electrodes 532, 532′. Two arrows indicate possible motion of the catheter (or lead) 530 where hysteresis may occur over a cardiac cycle. For example, a systolic path may differ from a diastolic path. An exemplary method may analyze hysteresis for any of a variety of purposes including assessing stability of an electrode of a catheter (or lead), assessing stability of a catheter (or lead), selection of a stimulation site, selection of a sensing site, diagnosis of cardiac condition, etc.

The exemplary method 540, as mentioned, includes the delivery block 542 for delivery of current, the measurement block 544 to measure potential in a field defined by the delivered current and the determination block 546 to determine position or motion based at least in part on the measured potential. According to such a method, position or motion during systole and/or diastole may be associated with electrical information or other information (e.g., biosensor, loading of a catheter or lead, intrinsic/paced activation, etc.). Alone, or in combination with other information, the position or motion information may be used for various assessments (e.g., stability assessments), selection of optimal stimulation site(s), determination of hemodynamic surrogates (e.g., surrogates to stroke volume, contractility, etc.), optimization of CRT, placement of leads, determination of pacing parameters (AV delay, VV delay, etc.), etc.

The system 500 may use one or more features of the aforementioned ENSITE® NAVX® system. For example, one or more pairs of electrodes (522, 522′, 524, 524′, 526, 526′ and optionally 528) may be used to define one or more dimensions by delivering an electrical signal or signals to a body and/or by sensing an electrical signal or signals. Such electrodes (e.g., patch electrodes) may be used in conjunction with one or more electrodes positioned in the body (e.g., the electrodes 532, 532′).

The exemplary system 500 may be used to track position or motion of one or more electrodes due to systolic function, diastolic function, respiratory function, etc. Electrodes may be positioned along the endocardium and/or epicardium during a scouting or mapping process for use in conjunction with electrical information. Such information may also be used alone, or in conjunction with other information (e.g., electrical information), for assessing stability of an electrode or electrodes for use in delivering a therapy or for identifying the optimal location of an electrode or electrodes for use in delivering a therapy. For example, a location may be selected for optimal stability, for optimal stimulation, for optimal sensing, or for other purposes.

With respect to stimulation, stimulation may be delivered to control cardiac mechanics (e.g., contraction of a chamber of the heart) and position or motion information may be acquired where such information is associated with the controlled cardiac mechanics. An exemplary selection process may identify the best stimulation site based on factors such as electrical activity, electromechanical delay, extent of motion, synchrony of motion where motion may be classified as motion due to systolic function or motion due to diastolic function. In general, motion information corresponds to motion of an electrode or electrodes (e.g., endocardial electrodes, epicardial electrodes, etc.) and may be related to motion of the heart or other physiology.

As described with respect to FIG. 5, a localization system can acquire position information for one or more electrodes on a lead or catheter. The ENSITE® NAVX® system can operate at a sampling frequency around 100 Hz (10 ms), which, for a cardiac rhythm of 60 bpm, allows for 100 samples per electrode per cardiac cycle. In various examples, sampling may be gated to occur over only a portion of a cardiac cycle. Gating may rely on fiducial markers such as peaks, gradients, crossings, etc., in an electrogram of heart activity. Other techniques for gating can include accelerometer techniques, impedance techniques, pressure techniques, flow techniques, etc. For example, an accelerometer signal slope above a threshold value (e.g., due to cardiac contraction or relaxation) can be used to commence acquisition of information or to terminate acquisition of information during a cardiac cycle. Such a technique may be repeated over multiple cardiac cycles with or without application of electrical stimuli, medication, body position changes, etc.

As described herein, for one or more electrodes, a localization system can provide four-dimensional information (e.g., x, y, z and time). The four-dimensional information describes a three-dimensional trajectory in space that can be analyzed or displayed in part, in whole or at one or more key points in time. As mentioned, various other types of information may be used to gate acquisition or to delineate points or segments of a trajectory. For example, information provided by a surface ECG, an intracardiac EGM (IEGM), or other biosignal can delineate a point or event such as QRS onset or pacing pulse or a segment (e.g., QRS complex, QT interval, etc.).

Where an electrode is position in a vessel of the heart such as a vein (e.g., coronary sinus (CS) vein or a tributary thereof), the trajectory of the electrode will follow cardiac motion of nearby myocardium. For example, a CS lead electrode will trace the path traversed by epicardium adjacent the CS or adjacent the particular CS tributary. If the lead position is stable in a branch, the trajectory for consecutive beats will typically remain within a bounded spatial volume; however, if the lead dislodges grossly, a shift in the CS lead electrode's position will be apparent in a display or analysis of the acquired information.

In various instances, depending on placement of electrodes that generate a localization field, respiration may affect accuracy of position data. For example, referring to FIG. 5, as a patient breathes, the torso changes shape, which can alter the alignment of the electrodes 522, 522′, 524, 524′, 526, 526′ and 528. Further, as respiration introduces air into the body, dielectric properties of media between electrodes of a directional pair may change. To account for the affects of respiration, an exemplary data acquisition technique may include an algorithm that compensates for respiratory motion. Alternatively, compensation of filtering may be performed after data acquisition, for example, using one or more algorithms that identify frequencies in data that are likely related to respiration and adjust the data (e.g., filter or normalize) to compensate for respiration. In other instances, respiration gating may be used during data acquisition, for example, akin to techniques used during acquisition of nuclear magnetic resonance data (e.g., NMR or MRI data). For example, beats to be included in a stability index metric may be gated to a particular portion of the respiratory cycle.

The ENSITE® NAVX® system includes a so-called “RespComp” algorithm that uses a combination of impedance between various pairs of patches, which create the localization field, as a measure of respiratory motion. In yet another alternative, motion of electrodes that are known to be stable can be used to ascertain respiratory motion. For example, position data with respect to time may have low frequency content (approximately 0.1 Hz to approximately 0.5 Hz) that can be due to respiration, which can be subtracted from the motion of the electrode of which stability is of interest.

Instantaneous fluid status, among other variables, can cause some drift in position as measured by a localization system such as the ENSITE® NAVX® system. An exemplary method can include a correction factor that accounts for fluid status drift, which may be found by comparing position of a stable electrode from one cycle to the next and applying any measured offset to an electrode of interest.

As described herein, for various vector metrics, subtraction techniques or other techniques may act to reduce or eliminate fluid status contributions or movement contributions caused by respiration, the heart in the body (e.g., within a localization field) or by patient movement (e.g., change in posture, etc.).

FIGS. 6, 7, 8 and 9 present three-dimensional maps of data acquired using the ENSITE® NAVX® system. Specifically, FIG. 6 shows two perspective views (A, B) of an isochronal map of the coronary sinus and several tributaries 610; FIG. 7 shows an isopotential map of the coronary sinus and several tributaries 710; FIG. 8 shows a path length map of the coronary sinus and several tributaries 810; and FIG. 9 shows a peak velocity map of the coronary sinus and several tributaries 910.

The isochronal map 610 of FIG. 6 shows activation time with respect to right atrial activation. The map 610 was generated by inserting a transvenous lead into the coronary sinus via the ostium, advancing the lead to various points and acquiring data. In the map 610, a comparison is readily made between the various tributaries of the coronary sinus where regions of late activation (e.g., greater than about 350 ms) may be identified. For example, the anterior vein is associated with quick activation. In CRT, the anterior vein may be a poor candidate for electrode placement as activity in the region surrounding the anterior vein is, in comparison, adequate. In contrast, the anterolateral vein and lateral vein indicate surrounding regions of late activation. As CRT aims to synchronize activation of the left ventricle, one of these two veins may be selected during a pre-implant planning process as candidates for exploration during an implant procedure (i.e., for placement of an electrode-bearing lead).

The isopotential map 710 of FIG. 7 shows peak-to-peak potentials for a cardiac cycle with respect to the coronary sinus and various tributaries. In the map 710, substantial portions of the anterior vein have the smallest peak-to-peak potentials while substantial portions of the lateral vein have the largest peak-to-peak potentials. In the map 710, a small peak-to-peak potential indicates little depolarization or activation. In contrast, a large peak-to-peak potential indicates significant depolarization and activation of myocardial tissue. According to the map 710, a pre-implant planning process may exclude at least some portions of the anterior vein and the anterolateral vein from further consideration as candidates for lead implant where such low peak-to-peak potentials indicate possibly damaged tissue that may not respond to electrical stimulation. However, regions that border possibly damaged tissue may be considered stimulation site candidates. For example, stimulation of healthy border tissue may improve cardiac performance (e.g., compensating for damaged tissue and possibly speeding recovery of damaged tissue, if possible). In another example, a pre-implant planning process that aims to place an electrode at or near healthy, active tissue, may consider the high peak-to-peak potential lateral branch regions shown in the map.

The path length map 810 of FIG. 8 is derived from electrode movement data. Specifically, the ENSITE® NAVX® system acquired electrode position data with respect to time, determined a path length for a cardiac cycle and mapped this data with respect to venous anatomy. Hence, the map 810 indicates extent of movement of the venous regions during a cardiac cycle. According to the map 810, the greatest motion occurs in the anterior vein while the least motion occurs in the anterolateral vein. In pre-implant planning, a clinician may exclude the anterior vein from consideration for placement of a pacing lead. While the units in the map 810 are presented in millimeters, such units may represent approximate measures depending on field characteristics of a localization system and whether field correction techniques are used.

The peak velocity map 910 of FIG. 9 is derived from electrode movement data (e.g., field compensated or uncompensated mm/s). Specifically, the ENSITE® NAVX® system acquired electrode position data with respect to time, determined a peak velocity for a cardiac cycle and mapped this data with respect to venous anatomy. Hence, the map 910 indicates peak velocity of movement of the venous regions during a cardiac cycle. According to the map 910, the greatest velocity occurs in the anterior vein while several regions have minimal velocity. Further, structural aspects of the heart may be inferred from such data. For example, where a high velocity appears proximate to a lower velocity, tissue associated with the lower velocity may be damaged or somewhat anchored (i.e., high peak velocity of adjacent tissue does not cause any significant movement). In pre-implant planning, a clinician may exclude the anterior vein from consideration for placement of a pacing lead. Using such a map, a clinician may choose to exclude a vein in close proximity to a kinetic tissue (e.g., where peak velocity is at or very close to zero), as pacing in an ischemic/infarct zone may provide negligible benefit to the patient. In such instances, a clinician may optionally confirm whether a region is associated with an ischemic or infarct zone based on a patient's previously collected imaging data (e.g., echo, MRI, CT, etc.) and, for example, ischemic cardiomyopathy history, if available.

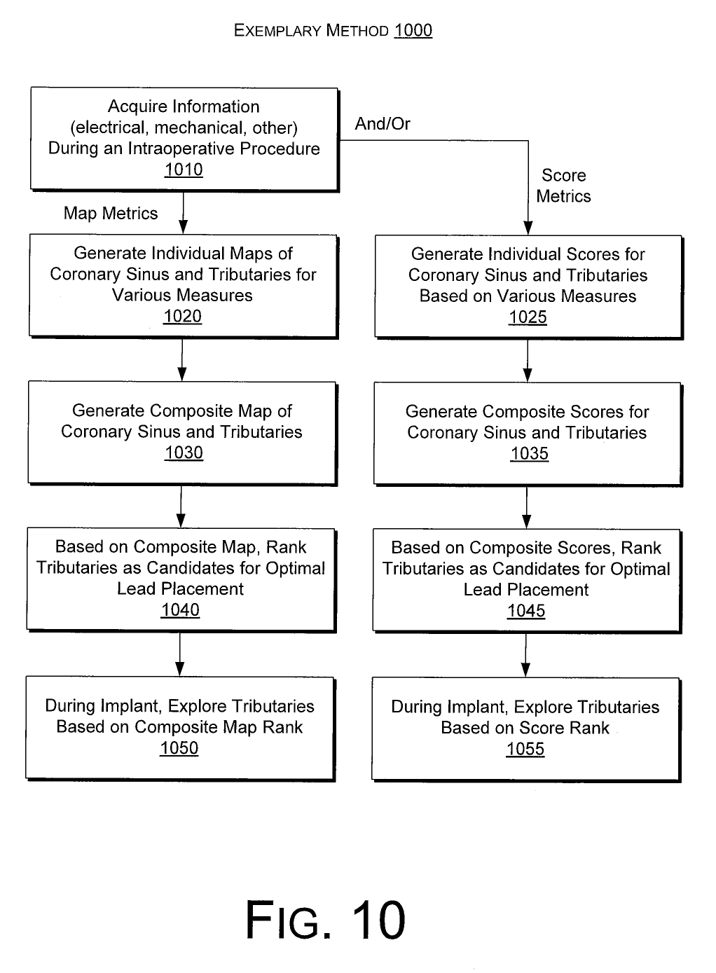

FIG. 10 shows an exemplary method 1000 that relies on mapping, scoring or optionally mapping and scoring to rank tributaries of the coronary sinus as likely candidates for optimal lead placement (e.g., for CRT). In an acquisition block 1010, during an intraoperative procedure, information is acquired using at least a localization system. Specifically, the acquisition block 1010 includes acquiring position information sufficient to localize information with respect to the coronary sinus to allow for generation of localized scores based at least in part on the information, generation of a map or maps to display at least some of the information or values derived from at least some of the information where such a map (or maps) includes anatomical markers or geometric boundaries of the coronary sinus and at least some of its tributaries.

In the example of FIG. 10, the method 1000 includes map-based ranking and score-based ranking. Such a method may optionally provide for hybrid map and score-based ranking. As described herein, one or more of these types of rankings may be performed.

According to the map-based ranking, a generation block 1020 includes generating individual maps of the coronary sinus and at least some of its tributaries for various measures (e.g., activation time, potential, displacement, velocity, acceleration, etc.). Another generation block 1030 includes generating one or more composite maps of the coronary sinus and at least some of its tributaries. Specifically, a composite map relies on at least two measures, which may be combined via a simple overlay or one or more other techniques. The generation of a composite map block 1030 may rely on graphics circuitry (e.g., a graphics card) where colors, shading, z-buffering, alpha blending, etc., may be used to generate a composite map. In a ranking block 1040, at least some of the tributaries are ranked as candidates for optimal lead placement based on the composite map. The ranking block 1040 may include ranking based on summing color or one or more other values over a region. For example, ranking may occur for regions where each region corresponds to a tributary that joins the coronary sinus. Where favorable measures are represented by higher intensity, a composite map may display intensities where ranking occurs based on intensity (higher intensity equals higher rank). Alternatively, where favorable measures are represented by lower intensity, lower intensity equals higher rank. In such examples, intensities may be summed and optionally rescaled for display in conjunction with anatomical markers or geometric representation of the coronary sinus and at least portions of tributaries thereto.

After ranking, the method 1000 includes an exploration block 1050 where, during an implant procedure, one or more tributaries are explored based on a map-based rank. For example, if a clinician determines that the top ranked tributary suffices (e.g., based on index being sufficiently superior to other tributaries), then the clinician may decide to implant a lead in the top ranked tributary and explore that tributary only to determine an optimal location for placement. In contrast, if the two top ranked tributaries appear similar on a composite map within some small margin (e.g., of color, intensity, shading, etc.), the clinician may decide to consider and explore both of these tributaries as candidates for lead placement.

For the score-based ranking, a generation block 1025 includes generating individual score for the coronary sinus and at least some of its tributaries. The score may correspond to various measures (e.g., activation time, potential, displacement, velocity, acceleration, etc.). Another generation block 1035 includes generating one or more composite scores for the coronary sinus and at least some of its tributaries. Specifically, a composite score relies on at least two measures, which may be input to an equation for calculation of a composite score. Such an equation or model may be based on information acquired from prior patients as to long-term results for a particular therapy. For example, where a patient has left-bundle branch block, an equation for calculating a composite score may include constants specific to this condition. Alternatively, where a patient has a diagnosed diastolic condition, other constants may be used. Accordingly, an equation may include various inputs (e.g., independent variables such as activation time, action potential, displacement, velocity, etc.) where constants are fit to information for a population of patients with a diagnosed condition. Such an equation may then provide a composite score as a dependent variable.

In a ranking block 1045, at least some of the tributaries are ranked as candidates for optimal lead placement based on composite scores. As mentioned, an equation may be used that includes inputs such as, for example, activation time, potential, path length and velocity to provide a value for an associated position. The tributaries may then be ranked by calculating an average value over regions. Such regions may be a selected according to a criterion or criteria (e.g., could be used for placement of a lead electrode). For example, the ranking may account for a lead electrode being placed at a sufficiently distal position (e.g., located at least one centimeter from a point of confluence with the coronary sinus). Other criteria may be introduced to appropriately rank (e.g., number of turns to reach a location, likely stability at location, etc.).

After ranking, in an exploration block 1050, during an implant procedure, one or more tributaries are explored based on rank. For example, if a clinician determines that the top ranked tributary suffices (e.g., based on a composite score being sufficiently superior to other tributaries), then the clinician may decide to implant a lead in the top ranked tributary and explore that tributary only to determine an optimal location for placement. In contrast, if the two top ranked tributaries have composite scores within some small margin, the clinician may decide to consider and explore both of these tributaries as candidates for lead placement.

As described herein, an exemplary method includes accessing cardiac information acquired via a catheter located at various positions in a coronary sinus of a patient where the cardiac information includes electrical information and mechanical information; mapping the electrical information and the mechanical information to a composite map where the composite map includes a geometric representation of at least the coronary sinus; and, based on the composite map, selecting a tributary of the coronary sinus as an optimal candidate for placement of a left ventricular lead. In such a method, the mapping may include mapping activation times where each of the activation times is a time defined in part by an intrinsic event or a paced event. Alternatively, or in addition to, the mapping may map action potentials where each action potential is a potential associated with an intrinsic event or a paced event. As explained, an exemplary method may include ranking the tributaries of the coronary sinus as candidates for placement of a left ventricular lead. An exemplary method may include mapping isochrones, isopotentials, displacement, velocity, etc. Mapping may map values or contours derived from values (e.g., using a spline fitting or other algorithm). Accordingly, a method may include overlaying contours for an electrical measure and contours for a mechanical measure. An exemplary method may include summing intensities where each intensity is an intensity derived from acquired or accessed electrical information or mechanical information. An exemplary method may include selecting a tributary or ranking tributaries by summing map values over a region or regions associated with a particular tributary (or tributaries) of the coronary sinus. An exemplary method may include selecting based on analyzing distances between the various positions and an anatomical feature (e.g., where the feature is a feature of the heart, a nerve or other anatomical feature). As described herein, various exemplary methods may be implemented in part by one or more computer-readable media having processor executable instructions to instruct a computing device.

As described herein, an exemplary method includes accessing cardiac information acquired via a catheter located at various positions in a coronary sinus of a patient where the cardiac information includes electrical information and mechanical information; calculating scores based on the cardiac information where each of the scores corresponds to the coronary sinus or a tributary of the coronary sinus; and, based on the scores, selecting a tributary of the coronary sinus as an optimal candidate for placement of a left ventricular lead. In such a method, the calculating may include calculating scores based at least in part on activation times where each of the activation times is a time defined in part by an intrinsic event or a paced event. Alternatively, or in addition to, the calculating may include calculating scores based at least in part on action potentials where each action potential is a potential associated with an intrinsic event or a paced event. As explained, an exemplary method may include ranking the tributaries of the coronary sinus as candidates for placement of a left ventricular lead. Various exemplary methods described herein may be implemented in part by one or more computer-readable media that have processor executable instructions to instruct a computing device.

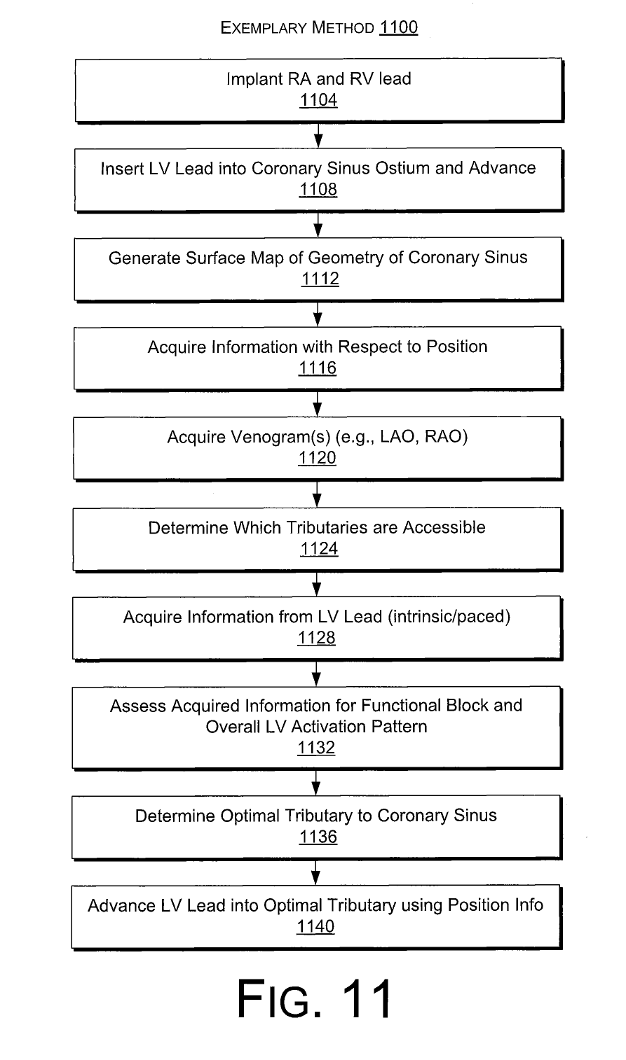

FIG. 11 shows an exemplary method 1100 for optimal placement of a left ventricular lead. In an implantation block 1104, a right atrial (RA) lead and a right ventricular (RV) apical lead are implanted as part of the standard CRT implant procedure. Next, in an insertion block 1108, a LV transvenous lead is inserted into the coronary sinus ostium and advanced to the end of the coronary sinus. In a generation block 1112, a surface map is generated for at least the coronary sinus as the LV lead is moved (e.g., to outline the geometric anatomy of the coronary sinus). In an acquisition block 1116, electrical information is then collected from one or more of the lead electrodes. For example, such information may allow for creating isochronal and isopotential maps to visualize electrical activation of regions surrounding the coronary sinus (see, e.g., FIGS. 6 and 7). To assist in locating a lead, an acquisition block 1120 includes acquiring one or more venograms (e.g., LAO and RAO views), which can be compared to corresponding views of the surface map geometry of block 1112. As indicated in block 1124, the method 1100 can rely on one or more venograms to provide a priori information about which coronary veins are accessible to the LV lead.

In an acquisition block 1128, information is acquired from at least the LV lead. The method 1100 may include acquiring such information while alternating between intrinsic rhythm and RV-only pacing, for example, while the LV lead is incrementally moved. In such an example, time from RV sense or pace to electrical activation of the LV lead electrode can be recorded at each position of the LV lead. Once the information is acquired per the acquisition block 1128, an assessment block 1132 assess the information, for example, to identify functional block, overall pattern of LV activation, etc. The assessment block 1132 may determine the site having the highest time from RV sense or pace to electrical activation and it may map various metrics for the coronary sinus and its accessible tributaries. As to functional block, differences between an intrinsic map and a RV paced map may help to identify functional block regions and help to understand overall LV activation pattern.

As indicated in the example of FIG. 11, the method 1100 includes an optimization block 1136 for determining the optimal tributary to the coronary sinus for placement of a LV lead. In an advancement block 1140, the LV lead is advanced into the optimal branch, which is deemed nearest or providing access to an optimal pacing site, which may be shown with respect to position information acquired using a localization system (e.g., optionally using the ENGUIDE™ locating signal feature of the ENSITE® NAVX® system).

In the method 1100, an optimization process may exclude one or more sites (or regions) where a criterion or criteria for selection are not met. By excluding one or more sites (or regions), an optimization algorithm may operate more expeditiously by reducing the number of options. Such an optimization process may include consideration of distance between a site or a region and one or more anatomical features. For example, if a site is too close to the ostium of the coronary sinus, the RA or the RV, that site may be excluded. In this example, one or more distance criteria may be used to determine whether a site or region should be excluded.

As described herein, one or more criteria may act to weight sites or regions. For example, consider a criterion that assigns a weight based on distance from an anatomical feature where the closer a site is to the feature, the smaller the weight or vice versa depending on whether proximity is beneficial. In another example, a distance range may be given where an optimal distance within the range is assigned the highest weight. Individual branches may be weighted based on an anatomical analysis, for example, coverage of the lateral wall of the left ventricle. In such an example, a branch that has more coverage (e.g., area) of the lateral wall may be assigned a higher weight. In another example, a general weighting may be post-lateral (highest), lateral (middle) and anterior and posterior (lowest). Another weighting scheme may assign a weight based on proximity to the apex versus the base of the heart. Where data has been acquired for a population or populations of patients, such data may be used to assign one or more weights to tributaries of the coronary sinus.

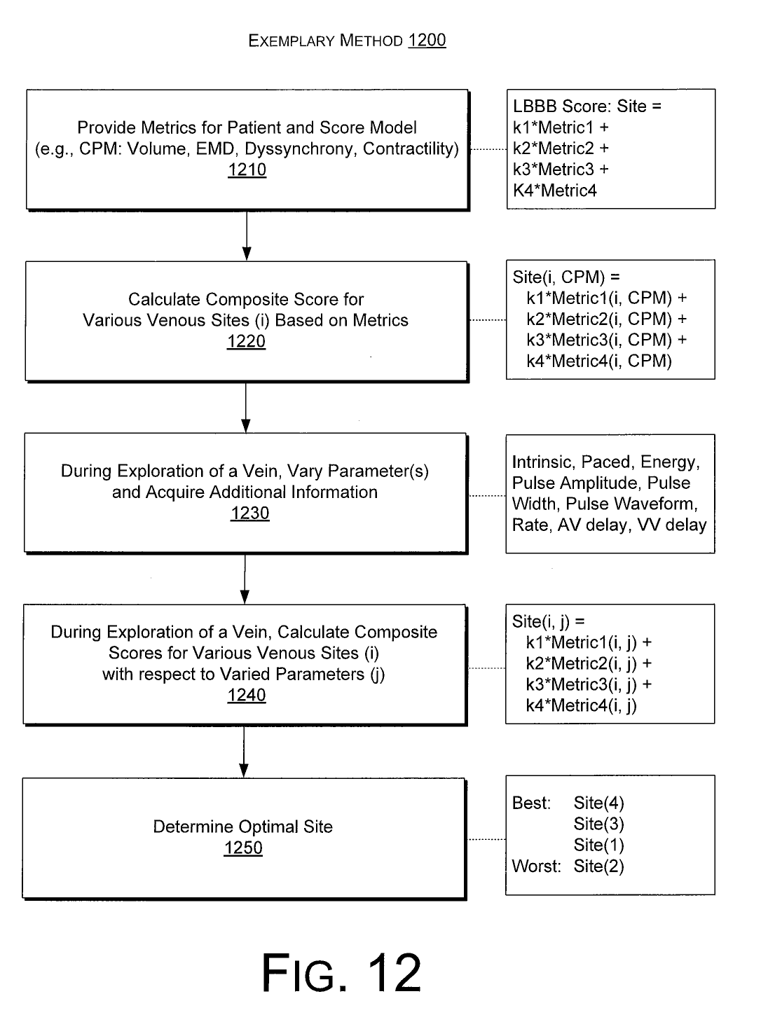

FIG. 12 shows an exemplary method 1200 for optimal positioning of a lead in a coronary vein. A provision block 1210 provides various metrics for a patient along with a score model. For example, to select the LV lead placement site longitudinally for a candidate vein, the method 1200 may be provided with metrics derived from an ENSITE® NAVX® system study. Specifically, such metrics may be a set of so-called Cardiac Performance Metrics (CPM). Such metrics may be based on motion data collected from the electrodes of a CRT setup (e.g., RA, RV and LV leads), plus any additional catheters that may have been inserted into the cardiac space. Mechanical metrics can include: volume estimators, electromechanical delays (EMD), dyssynchrony measures, and contractility. The goal would be to maximize the volume estimators, minimize EMDsd minimize dyssynchrony measures. As shown in the example of FIG. 12, a score model is also provided where the model depends on a diagnosis for a patient (e.g., left bundle branch block “LBBB”). Such a model may include dependent variables and constants determined from data acquired for a population of patients with LBBB. While the model shown is linear with three dependent variables and three constants, a model may be non-linear, include fewer or more variables, etc.

In the example of FIG. 12, the method 1200 includes a calculation block 1220 that calculates a composite score for various venous sites. For example, the following equation may be used to determine a composite score based on various metrics (e.g., CPM metrics):

Site(i)=k1*Volume+k2*EMD+k3*Dyssynchrony+k4*Contractility

where i represents a series of site (e.g., i=[base . . . apex]) and where k1, k2, k3 and k4 are weighted constants.

The foregoing equation may generate various scores where an optimal site for LV lead placement is equal to the minimum or maximum of the score (e.g., depending on the weights). The foregoing approach may be used to target different 2nd and 3rd order coronary sinus branches off a main branch that was identified in a prior step.

As shown in FIG. 12, the method 1200 includes an acquisition block 1230 that acquires additional information during exploration while parameters or conditions are being varied. For example, the block 1230 may iterate various rates, AV delays, and VV delays (or other values) while measuring information (e.g., underlying dependent variables) at various locations within a selected or candidate branch. Such an approach can be taken to enhance response to therapy or to achieve a more detailed composite score (e.g., reflecting a score for a particular therapy such as CRT). As indicated in a calculation block 1240, the information acquired during exploration per the block 1230 is used to calculate composite scores during exploration of a vein (e.g., optionally in a manner dependent on the varied parameters or conditions). For example, two nearby sites within a branch could have similar scores during intrinsic rhythm while their scores may separate upon the addition of an RV pace component; alternatively, modulation of AV delay or VV delay may identify a better candidate electrode position. In the case of a multi-electrode lead, information can be used to guide a programmed change in a pacing vector (electronic repositioning and/or multisite LV pacing) upon increased heart rate or other situations. As indicated by a determination block 1250, upon an assessment of the scores and conditions giving rise to the scores, the method 1200 may determine an optimal site for a LV lead or electrode thereof (e.g., to indicate a best site or optionally provide a rank for each site). The method 1200 may optionally include one or more criteria or weights as described with respect to the method 1100 of FIG. 11.

As described herein, an exemplary method includes accessing a ranking of tributaries of the coronary sinus where the ranking ranks the tributaries as candidates for placement of a left ventricular lead; selecting the highest ranked tributary; navigating the left ventricle lead into the selected tributary; acquiring information via the left ventricle lead for various locations in the selected tributary; mapping the acquired information or one or more metrics derived from the acquired information to a map where the map includes a geometric representation of at least a portion of the selected tributary; based on the map, optimally placing the left ventricular lead in the selected tributary. Such a method may include calculating a score for each of a plurality of sites as part of a mapping process and placing the left ventricular lead in the selected tributary based at least in part on the scores. As mentioned with respect to the method 1200 of FIG. 12, an information acquisition process may include altering one or more of pacing energy, pacing rate, atrio-ventricular delay, interventricular delay, etc. As described herein, after placing a lead in an optimal location, an exemplary method may include connecting the lead to an implantable device configured for delivery of cardiac resynchronization therapy. For example, during navigation, a lead may be connected to an external device (e.g., localization system, PSA, etc.). Specifically, once the optimal location is determined, the lead may then be detached from the external device and connected to an implantable device, which may be already implanted or thereafter implanted.

As mentioned, an exemplary method may include determining one or more distances (e.g., distance metrics). For example, a method may include mapping distance metrics based at least in part on distances between the various locations and an anatomical feature. In this example, the anatomical feature may be a feature of the heart such as, but not limited to, the right atrium, the right ventricle, the ostium of the coronary sinus, a valve of the heart, the apex of the heart and the base of the heart. In another example, an anatomical feature may be a nerve, such as, but not limited to, a phrenic nerve (e.g., to avoid phrenic nerve stimulation or to optionally stimulate the phrenic nerve, for example, as part of a respiratory therapy such as a sleep apnea therapy).

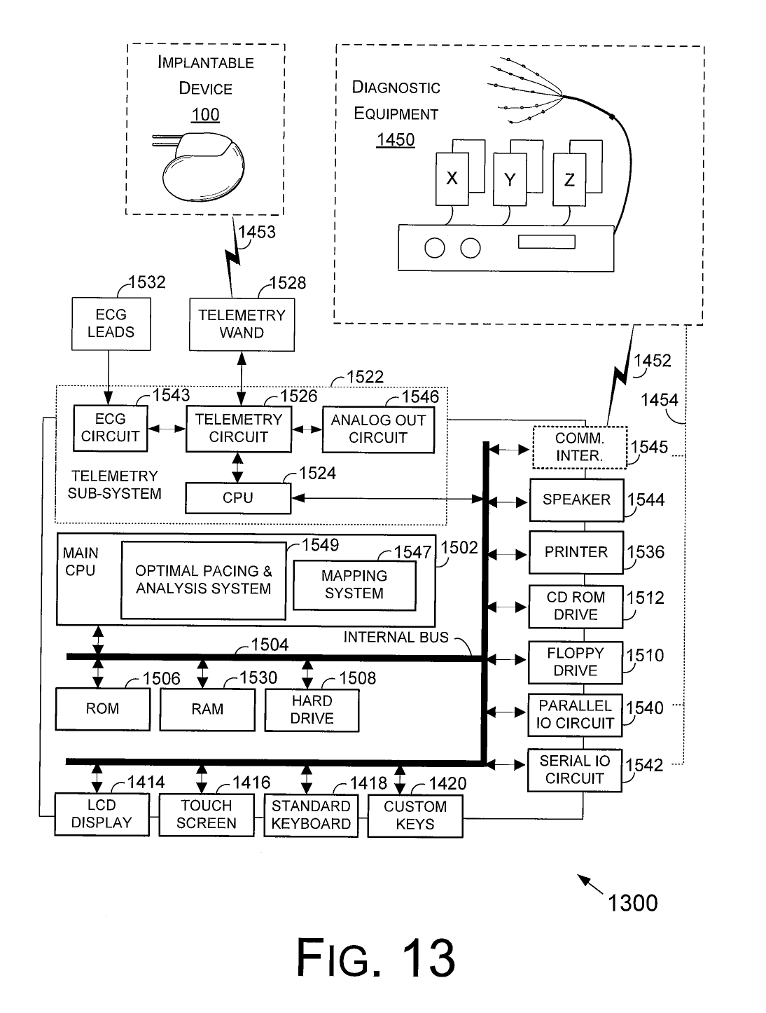

As described herein, the method 1200 may be optionally performed using a robotic system. For example, a robotic system may be programmed with a score model and a list of parameters or conditions to vary as well as a number of sites to investigate. To initiate the robotic exploration, a clinician may position a lead in a tributary and then allow the robotic system to maneuver the lead (e.g., a few centimeters) forward, backward, etc., until it determines an optimal site. Depending on the number of sites investigated and variation in parameters or conditions, such a process may be performed in a matter of minutes. For example, where four sites are investigated in a selected vein and tested with intrinsic and paced activation, the latter for three VV delays, with 10 acquisitions per variation, for a heart rate of about 60 bpm, acquisition and analysis for the 16 combinations of the process may take around 5 minutes. As described herein, the exemplary external programmer of FIG. 13 optionally includes a robotic mechanism to maneuver a lead in a vein and associated exemplary control logic to perform an acquisition and analysis process to arrive at an optimal site.

Further details on vector-magnitude based metrics are provided in U.S. patent application Ser. No. 12/621,373 (assigned in its entirety to Pacesetter, Inc.), titled “Cardiac Resynchronization Therapy Optimization Using Vector Measurements Obtained from Realtime Electrode Position Tracking,” the disclosure of which is hereby incorporated by reference.

Further details on area based metrics and volume based metrics are provided in U.S. patent application Ser. No. 12/398,460 (assigned in its entirety to Pacesetter, Inc.), titled “Cardiac Resynchronization Therapy Optimization Using Parameter Estimation from Realtime Electrode Motion Tracking,” the disclosure of which is hereby incorporated by reference.

Further details on mechanical dyssynchrony based metrics are provided in U.S. patent application Ser. No. 12/476,043 (assigned in its entirety to Pacesetter, Inc.), titled “Cardiac Resynchronization Therapy Optimization Using Mechanical Dyssynchrony and Shortening Parameters from Realtime Electrode Motion Tracking,” the disclosure of which is hereby incorporated by reference.

Further details on electrical and mechanical activation based metrics are provided in U.S. patent application Ser. No. 12/416,771 (assigned in its entirety to Pacesetter, Inc.), titled “Cardiac Resynchronization Therapy Optimization Using Electromechanical Delay from Realtime Electrode Motion Tracking,” the disclosure of which is hereby incorporated by reference.

Details on IEGM metrics corresponding to myocardial infarction and scarring are provided in U.S. patent application Ser. No. 12/639,788 (assigned in its entirety to Pacesetter, Inc.), titled “Methods to Identify Damaged or Scarred Tissue Based on Position Information and Physiological Information,” the disclosure of which is hereby incorporated by reference. Details on energy drain metrics corresponding to myocardial infarction and scarring are provided in U.S. patent application Ser. No. 12/553,413 (assigned in its entirety to Pacesetter, Inc.), titled “Pacing, Sensing and Other Parameter Maps Based on Localization System Data,” the disclosure of which is hereby incorporated by reference. Details on stability metrics corresponding to myocardial infarction and scarring are provided in U.S. patent application Ser. No. 12/562,003 (assigned in its entirety to Pacesetter, Inc.), titled “Electrode and Lead Stability Indexes and Stability Maps Based on Localization System Data,” the disclosure of which is hereby incorporated by reference.

### Exemplary External Programmer

FIG. 13 illustrates pertinent components of an external programmer 1300 for use in programming an implantable medical device 100 (see, e.g., FIGS. 1 and 2). The external programmer 1300 optionally receives information from other diagnostic equipment 1450, which may be a computing device capable of acquiring motion information related to cardiac mechanics. For example, the equipment 1450 may include a computing device to deliver current and to measure potentials using a variety of electrodes including at least one electrode positionable in the body (e.g., in a vessel, in a chamber of the heart, within the pericardium, etc.). Equipment may include a lead for chronic implantation or a catheter for temporary implantation in a patient's body. Equipment may allow for acquisition of respiratory motion and aid the programmer 1300 in distinguishing respiratory motion from cardiac.

Briefly, the programmer 1300 permits a clinician or other user to program the operation of the implanted device 100 and to retrieve and display information received from the implanted device 100 such as IEGM data and device diagnostic data. Where the device 100 includes a module such as the position/metrics module 239, then the programmer 1300 may instruct the device 100 to measure potentials associated with position or to determine metrics and to communicate such information to the programmer via a communication link 1453. The programmer 1300 may also instruct a device or diagnostic equipment to deliver current to generate one or more potential fields within a patient's body where the implantable device 100 may be capable of measuring potentials associated with the field(s).

The external programmer 1300 may be configured to receive and display ECG data from separate external ECG leads 1532 that may be attached to the patient. The programmer 1300 optionally receives ECG information from an ECG unit external to the programmer 1300. The programmer 1300 may use techniques to account for respiration.

Depending upon the specific programming, the external programmer 1300 may also be capable of processing and analyzing data received from the implanted device 100 and from ECG leads 1532 to, for example, render diagnosis as to medical conditions of the patient or to the operations of the implanted device 100. As noted, the programmer 1300 is also configured to receive data representative of conduction time delays from the atria to the ventricles and to determine, therefrom, an optimal or preferred configuration for pacing. Further, the programmer 1300 may receive information such as ECG information, IEGM information, information from diagnostic equipment, etc., and determine one or more metrics for optimizing therapy.

Considering the components of programmer 1300, operations of the programmer are controlled by a CPU 1502, which may be a generally programmable microprocessor or microcontroller or may be a dedicated processing device such as an application specific integrated circuit (ASIC) or the like. Software instructions to be performed by the CPU 1502 are accessed via an internal bus 1504 from a read only memory (ROM) 1506 and random access memory 1530. Additional software may be accessed from a hard drive 1508, floppy drive 1510, and CD ROM drive 1512, or other suitable permanent or removable mass storage device. Depending upon the specific implementation, a basic input output system (BIOS) is retrieved from the ROM 1506 by CPU 1502 at power up. Based upon instructions provided in the BIOS, the CPU 1502 “boots up” the overall system in accordance with well-established computer processing techniques.

Once operating, the CPU 1502 displays a menu of programming options to the user via an LCD display 1414 or other suitable computer display device. To this end, the CPU 1502 may, for example, display a menu of specific programming parameters of the implanted device 100 to be programmed or may display a menu of types of diagnostic data to be retrieved and displayed. In response thereto, the clinician enters various commands via either a touch screen 1416 overlaid on the LCD display or through a standard keyboard 1418 supplemented by additional custom keys 1420, such as an emergency VVI (EVVI) key. The EVVI key sets the implanted device to a safe VVI mode with high pacing outputs. This ensures life sustaining pacing operation in nearly all situations but by no means is it desirable to leave the implantable device in the EVVI mode at all times.

With regard to mapping of metrics (e.g., for patterns of conduction), the CPU 1502 includes a 3-D mapping system 1547 and an associated data analysis system 1549. The systems 1547 and 1549 may receive position information and physiological information from the implantable device 100 and/or diagnostic equipment 1450. The data analysis system 1549 optionally includes control logic to associate information and to make one or more conclusions based on metrics, for example, as indicated in FIG. 3 for planning an implant procedure or, more generally, to optimize delivery of therapy (e.g., to optimize a pacing configuration).

Where information is received from the implanted device 100, a telemetry wand 1528 may be used. Other forms of wireless communication exist as well as forms of communication where the body is used as a “wire” to communicate information from the implantable device 100 to the programmer 1300.

If information is received directly from diagnostic equipment 1450, any appropriate input may be used, such as parallel IO circuit 1540 or serial IO circuit 1542. Motion information received via the device 100 or via other diagnostic equipment 1450 may be analyzed using the mapping system 1547. In particular, the mapping system 1547 (e.g., control logic) may identify positions within the body of a patient and associate such positions with one or more electrodes where such electrodes may be capable of delivering stimulation energy to the heart, performing other actions or be associated with one or more sensors.

A communication interface 1545 optionally allows for wired or wireless communication with diagnostic equipment 1450 or other equipment (e.g., equipment to ablate or otherwise treat a patient). The communication interface 1545 may be a network interface connected to a network (e.g., intranet, Internet, etc.).

A map or model of cardiac information may be displayed using display 1414 based, in part, on 3-D heart information and optionally 3-D torso information that facilitates interpretation of information. Such 3-D information may be input via ports 1540, 1542, 1545 from, for example, a database, a 3-D imaging system, a 3-D location digitizing apparatus (e.g., stereotactic localization system with sensors and/or probes) capable of digitizing the 3-D location. While 3-D information and localization are mentioned, information may be provided with fewer dimensions (e.g., 1-D or 2-D). For example, where motion in one dimension is insignificant to one or more other dimensions, then fewer dimensions may be used, which can simplify procedures and reduce computing requirements of a programmer, an implantable device, etc. The programmer 1300 optionally records procedures and allows for playback (e.g., for subsequent review). For example, a heart map and all of the electrical activation data, mechanical activation data, etc., may be recorded for subsequent review, perhaps if an electrode needs to be repositioned or one or more other factors need to be changed (e.g., to achieve an optimal configuration). Electrodes may be lead based or non-lead based, for example, an implantable device may operate as an electrode and be self powered and controlled or be in a slave-master relationship with another implantable device (e.g., consider a satellite pacemaker, etc.). An implantable device may use one or more epicardial electrodes.

Once all pacing leads are mounted and all pacing devices are implanted (e.g., master pacemaker, satellite pacemaker, biventricular pacemaker), the various devices are optionally further programmed.

The telemetry subsystem 1522 may include its own separate CPU 1524 for coordinating the operations of the telemetry subsystem. In a dual CPU system, the main CPU 1502 of programmer communicates with telemetry subsystem CPU 1524 via internal bus 1504. Telemetry subsystem additionally includes a telemetry circuit 1526 connected to telemetry wand 1528, which, in turn, receives and transmits signals electromagnetically from a telemetry unit of the implanted device. The telemetry wand is placed over the chest of the patient near the implanted device 100 to permit reliable transmission of data between the telemetry wand and the implanted device.

Typically, at the beginning of the programming session, the external programming device 1300 controls the implanted device(s) 100 via appropriate signals generated by the telemetry wand to output all previously recorded patient and device diagnostic information. Patient diagnostic information may include, for example, motion information (e.g., cardiac, respiratory, etc.) recorded IEGM data and statistical patient data such as the percentage of paced versus sensed heartbeats. Device diagnostic data includes, for example, information representative of the operation of the implanted device such as lead impedances, battery voltages, battery recommended replacement time (RRT) information and the like.

Data retrieved from the implanted device(s) 100 can be stored by external programmer 1300 (e.g., within a random access memory (RAM) 1530, hard drive 1508, within a floppy diskette placed within floppy drive 1510). Additionally, or in the alternative, data may be permanently or semi-permanently stored within a compact disk (CD) or other digital media disk, if the overall system is configured with a drive for recording data onto digital media disks, such as a write once read many (WORM) drive. Where the programmer 1300 has a communication link to an external storage device or network storage device, then information may be stored in such a manner (e.g., on-site database, off-site database, etc.). The programmer 1300 optionally receives data from such storage devices.

A typical procedure may include transferring all patient and device diagnostic data stored in an implanted device 100 to the programmer 1300. The implanted device(s) 100 may be further controlled to transmit additional data in real time as it is detected by the implanted device(s) 100, such as additional motion information, IEGM data, lead impedance data, and the like. Additionally, or in the alternative, telemetry subsystem 1522 receives ECG signals from ECG leads 1532 via an ECG processing circuit 1534. As with data retrieved from the implanted device 100, signals received from the ECG leads are stored within one or more of the storage devices of the programmer 1300. Typically, ECG leads output analog electrical signals representative of the ECG. Accordingly, ECG circuit 1534 includes analog to digital conversion circuitry for converting the signals to digital data appropriate for further processing within programmer 1300. Depending upon the implementation, the ECG circuit 1543 may be configured to convert the analog signals into event record data for ease of processing along with the event record data retrieved from the implanted device. Typically, signals received from the ECG leads 1532 are received and processed in real time.

Thus, the programmer 1300 is configured to receive data from a variety of sources such as, but not limited to, the implanted device 100, the diagnostic equipment 1450 and directly or indirectly via external ECG leads (e.g., subsystem 1522 or external ECG system). The diagnostic equipment 1450 includes wired 1454 and/or wireless capabilities 1452 which optionally operate via a network that includes the programmer 1300 and the diagnostic equipment 1450 or data storage associated with the diagnostic equipment 1450.

Data retrieved from the implanted device(s) 100 typically includes parameters representative of the current programming state of the implanted devices. Under the control of the clinician, the external programmer displays the current programming parameters and permits the clinician to reprogram the parameters. To this end, the clinician enters appropriate commands via any of the aforementioned input devices and, under control of CPU 1502, the programming commands are converted to specific programming parameters for transmission to the implanted device 100 via telemetry wand 1528 to thereby reprogram the implanted device 100 or other devices, as appropriate.

Prior to reprogramming specific parameters, the clinician may control the external programmer 1300 to display any or all of the data retrieved from the implanted device 100, from the ECG leads 1532, including displays of ECGs, IEGMs, statistical patient information (e.g., via a database or other source), diagnostic equipment 1450, etc. Any or all of the information displayed by programmer may also be printed using a printer 1536.

A wide variety of parameters may be programmed by a clinician. In particular, for CRT, the AV delay and the VV delay of the implanted device(s) 100 are set to optimize cardiac function. In one example, the VV delay is first set to zero while the AV delay is adjusted to achieve the best possible cardiac function, optionally based on motion information. Then, VV delay may be adjusted to achieve still further enhancements in cardiac function.

Programmer 1300 optionally includes a modem to permit direct transmission of data to other programmers via the public switched telephone network (PSTN) or other interconnection line, such as a T1 line or fiber optic cable. Depending upon the implementation, the modem may be connected directly to internal bus 1504 may be connected to the internal bus via either a parallel port 1540 or a serial port 1542.

Other peripheral devices may be connected to the external programmer via the parallel port 1540, the serial port 1542, the communication interface 1545, etc. Although one of each is shown, a plurality of input output (IO) ports might be provided. A speaker 1544 is included for providing audible tones to the user, such as a warning beep in the event improper input is provided by the clinician. Telemetry subsystem 1522 additionally includes an analog output circuit 1546 for controlling the transmission of analog output signals, such as IEGM signals output to an ECG machine or chart recorder.

With the programmer 1300 configured as shown, a clinician or other user operating the external programmer is capable of retrieving, processing and displaying a wide range of information received from the ECG leads 1532, from the implanted device 100, the diagnostic equipment 1450, etc., and to reprogram the implanted device 100 or other implanted devices if needed. The descriptions provided herein with respect to FIG. 13 are intended merely to provide an overview of the operation of programmer and are not intended to describe in detail every feature of the hardware and software of the device and is not intended to provide an exhaustive list of the functions performed by the device 1300. Other devices, particularly computing devices, may be used.

### CONCLUSION

Although exemplary methods, devices, systems, etc., have been described in language specific to structural features and/or methodological acts, it is to be understood that the subject matter defined in the appended claims is not necessarily limited to the specific features or acts described. Rather, the specific features and acts are disclosed as exemplary forms of implementing the claimed methods, devices, systems, etc.

1. A method comprising:
accessing cardiac information acquired via a catheter located at various positions in a coronary sinus of a patient wherein the cardiac information comprises electrical information and mechanical information;
calculating scores based on the cardiac information wherein each of the scores corresponds to the coronary sinus or a tributary of the coronary sinus; and,
based on the scores, selecting a tributary of the coronary sinus as an optimal candidate for placement of a left ventricular lead.

2. The method of claim 1 wherein calculating comprises calculating scores based at least in part on activation times wherein each of the activation times comprises a time defined in part by an intrinsic event or a paced event.

3. The method of claim 1 wherein calculating comprises calculating scores based at least in part on action potentials wherein each action potential comprises a potential associated with an intrinsic event or a paced event.

4. The method of claim 1 wherein calculating comprises calculating scores based at least in part on distances between the various positions and an anatomical feature.

5. The method of claim 4 wherein the anatomical feature comprises a feature of the heart.

6. The method of claim 4 wherein the anatomical feature comprises a nerve.

7. The method of claim 1 further comprising ranking the tributaries of the coronary sinus as candidates for placement of a left ventricular lead.

8. The method of claim 1 further comprising mapping the scores to a map wherein the map includes a geometric representation of at least the coronary sinus.

9. The method of claim 1 further comprising mapping the electrical information to a map wherein the map includes a geometric representation of at least the coronary sinus.

10. The method of claim 1 further comprising mapping the mechanical information to a map wherein the map includes a geometric representation of at least the coronary sinus.

11. The method of claim 1 further comprising mapping isochrones or isopotentials to a map wherein the map includes a geometric representation of at least the coronary sinus.

12. The method of claim 1 further comprising mapping displacement or velocity to a map wherein the map includes a geometric representation of at least the coronary sinus.

13. One or more computer-readable media comprising processor executable instructions to instruct a computing device to:
access cardiac information acquired via a catheter located at various positions in a coronary sinus of a patient wherein the cardiac information comprises electrical information and mechanical information;
calculate scores based on the cardiac information wherein each of the scores corresponds to the coronary sinus or a tributary of the coronary sinus; and,
based on the scores, select a tributary of the coronary sinus as an optimal candidate for placement of a left ventricular lead.

14. A method comprising:
accessing cardiac information acquired via a catheter located at various positions in a coronary sinus of a patient wherein the cardiac information comprises electrical information and mechanical information;
mapping the electrical information and the mechanical information to a composite map wherein the composite map includes a geometric representation of at least the coronary sinus; and,
based on the composite map, selecting a tributary of the coronary sinus as an optimal candidate for placement of a left ventricular lead.

15. The method of claim 14 wherein mapping comprises mapping activation times wherein each of the activation times comprises a time defined in part by an intrinsic event or a paced event.

16. The method of claim 14 wherein mapping comprises mapping action potentials wherein each action potential comprises a potential associated with an intrinsic event or a paced event.

17. The method of claim 14 further comprising ranking the tributaries of the coronary sinus as candidates for placement of a left ventricular lead.

18. The method of claim 14 wherein the mapping comprises mapping isochrones or isopotentials.

19. The method of claim 14 wherein the mapping comprises mapping displacement or velocity.

20. The method of claim 14 wherein the mapping comprises overlaying contours for an electrical measure and contours for a mechanical measure.

21. The method of claim 14 wherein the mapping comprises summing intensities wherein each intensity comprises an intensity derived from the electrical information or the mechanical information.

22. The method of claim 14 wherein the selecting comprises summing map values over a region associated with a particular tributary of the coronary sinus.

23. The method of claim 14 wherein the selecting comprises analyzing distances between the various positions and an anatomical feature.

24. One or more computer-readable media comprising processor executable instructions to instruct a computing device to:
access cardiac information acquired via a catheter located at various positions in a coronary sinus of a patient wherein the cardiac information comprises electrical information and mechanical information;
map the electrical information and the mechanical information to a composite map wherein the composite map includes a geometric representation of at least the coronary sinus; and,
based on the composite map, select a tributary of the coronary sinus as an optimal candidate for placement of a left ventricular lead.

25. A method comprising:
accessing a ranking of tributaries of the coronary sinus wherein the ranking ranks the tributaries as candidates for placement of a left ventricular lead;
acquiring information via a left ventricle lead for various locations in a selected tributary;
mapping the acquired information, or one or more metrics derived from the acquired information, to a map wherein the map includes a geometric representation of at least a portion of the selected tributary;
based on the map, optimally placing the left ventricular lead in the selected tributary.

26. The method of claim 25 wherein mapping comprises calculating a score for each of a plurality of sites.

27. The method of claim 26 wherein optimally placing comprises placing the left ventricular lead in the selected tributary based at least in part on the scores.

28. The method of claim 25 wherein mapping comprises mapping distance metrics based at least in part on distances between the various locations and an anatomical feature.

29. The method of claim 28 wherein the anatomical feature comprises a feature of the heart.

30. The method of claim 25 wherein acquiring information comprises altering at least one member selected from a group consisting of pacing energy, pacing rate, atrio-ventricular delay and interventricular delay.

31. One or more computer-readable media comprising processor executable instructions to instruct a computing device to:
access a ranking of tributaries of the coronary sinus wherein the ranking ranks the tributaries as candidates for placement of a left ventricular lead;
select the highest ranked tributary;
acquire information via the left ventricle lead for various locations in the selected tributary;
map the acquired information or one or more metrics derived from the acquired information to a map wherein the map includes a geometric representation of at least a portion of the selected tributary;
based on the map, optimally place the left ventricular lead in the selected tributary.
